(19) United States
(12) Patent Application Publication
(10) Pub. No.: US 2025/0204857 A1
(43) Pub. Date: Jun. 26, 2025

(54) IMPLANTABLE CARDIAC MONITOR

(71) Applicant: Oracle Health, Inc., Safety Harbor, FL (US)

(72) Inventor: Jaeson Bang, Safety Harbor, FL (US)

(21) Appl. No.: 18/846,871

(22) PCT Filed: Mar. 21, 2023
(86) PCT No.: PCT/US2023/015734

§ 371 Date: Sep. 13, 2024

Related U.S. Application Data
(60) Provisional application No. 63/321,936, filed on Mar. 21, 2022.

(51) Int. Cl. A61B 5/00

(52) U.S. Cl. CPC A61B 5/686; A61B 2560/0219; A61B 2560/045

(57) ABSTRACT

A patient diagnostic system comprises at least one implantable device configured to record one or more patient physiologic parameters. The at least one implantable device comprising one or more of each of the following implantable sensors: an accelerometer; a pressure sensor; a temperature sensor; an acoustic sensor, and a pair of electrodes. At least one external device configured to produce data can also be included in the system.

### RELATED APPLICATIONS

This application claims benefit to U.S. Provisional Application Ser. No. 63/321,936 (Docket No.: OCL-002-PR1), titled “IMPLANTABLE CARDIAC MONITOR”, filed Mar. 21, 2022, the content of which is incorporated by reference in its entirety.

This application is related to U.S. Provisional Application Ser. No. 62/853,899 (Docket No.: OCL-001-PR1), titled “IMPLANTABLE CARDIAC MONITOR”, filed May 29, 2019, the content of which is incorporated by reference in its entirety.

This application is related to U.S. patent application Ser. No. 17/611,335 (Docket No.: OCL-001-US), titled “IMPLANTABLE CARDIAC MONITOR”, filed Nov. 15, 2021, United States Publication Number 2022-0192600, published Jun. 23, 2022, the content of which is incorporated by reference in its entirety.

This application is related to International PCT Patent Application Serial Number PCT/US2020/035171 (Docket No.: OCL-001-PCT), titled “IMPLANTABLE CARDIAC MONITOR”, filed May 29, 2020, United States Publication Number WO2020/243463, published Dec. 3, 2020, the content of which is incorporated by reference in its entirety.

This application is related to U.S. Provisional Application Ser. No. 63/259,912 (Docket No.: OCL-003-PR1), titled “IMPLANTABLE ANTENNA AND SENSOR CONFIGURATIONS”, filed Jul. 28, 2021, the content of which is incorporated by reference in its entirety.

This application is related to U.S. patent application Ser. No. 17/443,899 (Docket No.: OCL-003-US), titled “IMPLANTABLE ANTENNA AND SENSOR CONFIGURATIONS”, filed Jul. 28, 2021, the content of which is incorporated by reference in its entirety.

This application is related to International PCT Patent Application Serial Number PCT/US2022/038653 (Docket No.: OCL-003-PCT), titled “IMPLANTABLE ANTENNA AND COEXISTING SENSOR CONFIGURATIONS”, filed Jul. 28, 2022, United States Publication Number WO2023/009707, published Feb. 2, 2023, the content of which is incorporated by reference in its entirety.

### FIELD OF THE INVENTIVE CONCEPTS

The present inventive concepts relate generally to a patient diagnostic system for detecting changes in patient vital signs over extended periods of time, and in particular a system including an implantable device for monitoring cardiac function.

### BACKGROUND

Heart failure (HF) hospital admissions cost the United States an estimated $21 billion per year. Nearly 5.7 million Americans have congestive heart failure (CHF), and approximately 500,000 new cases are diagnosed each year. Approximately 1.2 million people are hospitalized for HF each year, and 1 in 5 people will develop HF in their lifetime (26 million worldwide). Fifty percent (50%) of the patients die within 5 years of diagnosis. Heart failure patients in general are segmented into two main groups. HFrEF (Reduced Ejection Fraction Heart Failure), and HFpEF (Preserved Ejection Fraction Heart Failure). Both groups suffer greatly from the disease state, and the HFrEF group are typically indicated for medical device therapy using Bi-Ventricular Defibrillators with clinically acceptable monitoring solutions.

However, the HFpEF population (Preserved Ejection Fraction Heart Failure) have no viable heart-monitoring options outside of a clinic. Current methods include blood pressure monitoring and volume control and rely on delayed and often inaccurate data (e.g. weight gain, and leg swelling). Patients in this population are often left with only two undesirable choices: call the clinic or go to the emergency room (ER).

Traditional vital sign measuring methods are not equipped to monitor trending changes over extended periods of time, nor are they properly equipped to monitor changes throughout the day and night for extended periods of time.

Existing commercial devices, such as prior art implantable cardiac monitors (e.g. Insertable Loop Recorders, ILR), monitor cardiac arrhythmias, but they are not able to monitor other important vital signs of the patient.

Syncope accounts for over 740,000 ER visits per year, and about 240,000 hospitalizations. However, half of those patients leave the hospital without a proper diagnosis being performed.

Existing wearable heart monitoring devices (e.g. including skin-attached patches) suffer from several undesirable limitations. For example, existing devices are: (1) unable to obtain data around the clock for extended periods of time (weeks, months, or years); (2) unable to obtain high quality data throughout the day and night for extended periods of time; and (3) difficult for a patient to be compliant for extended periods of time (months). Moreover, conventional implantable cardiac monitors are limited in focus to detecting arrhythmias (atrial fibrillation, slow heart beats, fast heart beats) caused by electrical conduction disturbances within the heart.

Currently available implantable cardiac monitors do not address potential mechanical issues of the patient, such as those related to filling and ejection sounds of the heart.

Currently available implantable devices do not address other important patient vital signs. The embodiments disclosed herein are aimed at overcoming these and other undesirable limitations in the art.

### SUMMARY

According to an aspect of the present inventive concepts, a system for producing a diagnostic a patient comprising at least one implantable device configured to record physiologic data of the patient, and at least one external device configured to: transmit power and/or data to the at least one implantable device; and/or receive data from the at least one implantable device. The system can be configured to produce a diagnostic result based on the recorded physiologic data.

In some embodiments, the at least one implantable device comprises one or more sensors configured to record the physiologic data of the patient. The one or more sensors can be configured to record patient data comprising two, three, or four of: heart sounds, patient movement, EEG, and patient temperature. The external device can be configured to receive the recorded patient data and analyze the received patient data. The one or more sensors can comprise at least an accelerometer and at least an electrode. The one or more sensors can comprise an accelerometer. The accelerometer can comprise a 3-axis accelerometer. The system can be configured to record heart sounds and to determine an origin of the recorded heart sounds based on signals recorded by the 3-axis accelerometer. The 3-axis accelerometer can be configured to record at least two, three, or four of: heart sounds of the patient, the activity level of the patient, body position of the patient, and/or body posture of the patient.

In some embodiments, the system comprises multiple sensors configured to record a first set of physiologic parameters. The sensors can record values of the first set of physiologic parameters that change within a time period of no more than 10 milliseconds, 7 milliseconds, 4 milliseconds, and/or 2 milliseconds. The sensors can record values of the first set of physiologic parameters that comprise changes in tissue motion that are less than or equal to 5 mm, 3 mm, 1.5 mm, and/or 0.5 mm. The diagnostic result can be based on the recorded changes to the first set of physiologic parameters.

In some embodiments, the system further comprises a 3-axis accelerometer configured to record vibrations of the heart of the patient and a set of one or more sensors configured to record one or more of: body activity; body posture; and/or body orientation data. The system can calculate heart sound data based on the recorded vibrations of the heart. The system can produce the diagnostic result based on the calculated heart sound data and the data recorded by the first set of one or more sensors.

In some embodiments, the system comprises a first set of one or more sensors configured to record heart sounds of the patient and a second set of one or more sensors configured to record an ECG of the patient. The first set of sensors and the second set of sensors can be configured to record changes in physiologic parameters that occur within a time period of no more than 10 milliseconds, 7 milliseconds, 4 milliseconds, and/or 2 milliseconds. The system can determine the electromechanical activation time and the left ventricular systolic time based on the recordings of the first set of sensors and the second set of sensors.

In some embodiments, the at least one implantable device comprises a power supply that is configured to provide sufficient energy to allow two-year use of the at least one implantable device.

In some embodiments, the at least one implantable device comprises at least a first implantable device and a second implantable device. The system can be configured to enhance the diagnostic result based on data collected from both the first implantable device and the second implantable device. The first implantable device can be configured to be implanted in the upper left chest of the patient, and the second implantable device can be configured to be implanted in the lower left abdomen of the patient. The system can be configured to record EKG data with a wide vector. The first implantable device can be configured to be implanted proximate a wrist of the patient and the second implantable device can be configured to be implanted at a location remote from the wrist of the patient. The system can be configured to record patient data with a wide vector.

In some embodiments, the external device is configured to: retrieve data from the at least one implantable device; transmit software update data to the implantable device; and/or recharge the at least one implantable device.

In some embodiments, the at least one implantable device comprises a magnetic sensor configured to detect the presence of an external magnet placed proximate the at least one implantable device, and the at least one implantable device is configured to change operational modes when the presence of the external magnet is detected.

The technology described herein, along with the attributes and attendant advantages thereof, will best be appreciated and understood in view of the following detailed description taken in conjunction with the accompanying drawings in which representative embodiments are described by way of example.

### INCORPORATION BY REFERENCE

All publications, patents, and patent applications mentioned in this specification are herein incorporated by reference to the same extent as if each individual publication, patent, or patent application was specifically and individually indicated to be incorporated by reference. The content of all publications, patents, and patent applications mentioned in this specification are herein incorporated by reference in their entirety for all purposes.

### BRIEF DESCRIPTION OF THE DRAWINGS

FIG. 1 illustrates a schematic view of an implantable cardiac monitoring device in communication with various other patient devices, consistent with the present inventive concepts.

FIGS. 1A-C illustrate ECG data, sound signature data (phonocardiogram, PCG) data, seismocardiography (SCG) data, and impedance cardiography (ICG, electrical and fluid) level data gathered by the systems and devices of the present inventive concepts.

FIGS. 2A-C illustrate patient devices displaying various physiological recordings based on data collected by the implantable cardiac monitor, consistent with the present inventive concepts.

FIG. 3 illustrates a schematic view of a patient diagnostic system, consistent with the present inventive concepts.

FIGS. 3A-C illustrates graphs of recorded cardiac function, consistent with the present inventive concepts.

FIGS. 3D-F illustrate heart sounds recorded from three human subjects, consistent with the present inventive concepts.

FIG. 4 illustrates a schematic view of a patient diagnostic system including multiple implanted sensing devices, consistent with the present inventive concepts.

FIG. 5 is a top view of two electronic assemblies of an implantable device, consistent with the present inventive concepts.

### DETAILED DESCRIPTION OF THE DRAWINGS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer, or section. Thus, a first limitation, element, component, region, layer, or section discussed below could be termed a second limitation, element, component, region, layer, or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

As used herein, the terms “operably attached”, “operably connected”, “operatively coupled” and similar terms related to attachment of components shall refer to attachment of two or more components that results in one, two, or more of: electrical attachment; fluid attachment; magnetic attachment; mechanical attachment; optical attachment; sonic attachment; and/or other operable attachment arrangements. The operable attachment of two or more components can facilitate the transmission between the two or more components of: power; signals; electrical energy; fluids or other flowable materials; magnetism; mechanical linkages; light; sound such as ultrasound; and/or other materials and/or components.

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g. a target tissue location), shall include components positioned near to the anatomical site, as well as components positioned in, on and/or within the anatomical site.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, and “prevention” shall include the acts of “reduce”, “reducing”, and “reduction”, respectively.

The term “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

The term “one or more”, where used herein can mean one, two, three, four, five, six, seven, eight, nine, ten, or more, up to any number.

The terms “and combinations thereof” and “and combinations of these” can each be used herein after a list of items that are to be included singly or collectively. For example, a component, process, and/or other item selected from the group consisting of: A; B; C; and combinations thereof, shall include a set of one or more components that comprise: one, two, three or more of item A; one, two, three or more of item B; and/or one, two, three, or more of item C.

In this specification, unless explicitly stated otherwise, “and” can mean “or”, and “or” can mean “and”. For example, if a feature is described as having A, B, or C, the feature can have A, B, and C, or any combination of A, B, and C. Similarly, if a feature is described as having A, B, and C, the feature can have only one or two of A, B, or C.

As used herein, when a quantifiable parameter is described as having a value “between” a first value X and a second value Y, it shall include the parameter having a value of: at least X, no more than Y, and/or at least X and no more than Y. For example, a length of between 1 and 10 shall include a length of at least 1 (including values greater than 10), a length of less than 10 (including values less than 1), and/or values greater than 1 and less than 10.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

As used herein, the terms “about” or “approximately” shall refer to ±30%.

As used herein, the term “threshold” refers to a maximum level, a minimum level, and/or range of values correlating to a desired or undesired state. In some embodiments, a system parameter is maintained above a minimum threshold, below a maximum threshold, within a threshold range of values, and/or outside a threshold range of values, such as to cause a desired effect (e.g. efficacious therapy) and/or to prevent or otherwise reduce (hereinafter “prevent”) an undesired event (e.g. a device and/or clinical adverse event). In some embodiments, a system parameter is maintained above a first threshold (e.g. above a first temperature threshold to cause a desired therapeutic effect to tissue) and below a second threshold (e.g. below a second temperature threshold to prevent undesired tissue damage). In some embodiments, a threshold value is determined to include a safety margin, such as to account for patient variability, system variability, tolerances, and the like. As used herein, “exceeding a threshold” relates to a parameter going above a maximum threshold, below a minimum threshold, within a range of threshold values and/or outside of a range of threshold values.

As described herein, “room pressure” shall mean pressure of the environment surrounding the systems and devices of the present inventive concepts. Positive pressure includes pressure above room pressure or simply a pressure that is greater than another pressure, such as a positive differential pressure across a fluid pathway component such as a valve. Negative pressure includes pressure below room pressure or a pressure that is less than another pressure, such as a negative differential pressure across a fluid component pathway such as a valve. Negative pressure can include a vacuum but does not imply a pressure below room pressure. As used herein, the term “vacuum” can be used to refer to a full or partial vacuum, or any negative pressure as described herein.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross-sectional area as the cross section of the component being described.

The terms “major axis” and “minor axis” of a component where used herein are the length and diameter, respectively, of the smallest volume hypothetical cylinder which can completely surround the component.

As used herein, the term “functional element” is to be taken to include one or more elements constructed and arranged to perform a function. A functional element can comprise a sensor and/or a transducer. In some embodiments, a functional element is configured to deliver energy and/or otherwise treat tissue (e.g. a functional element configured as a treatment element). Alternatively or additionally, a functional element (e.g. a functional element comprising a sensor) can be configured to record one or more parameters, such as a patient physiologic parameter; a patient anatomical parameter (e.g. a tissue geometry parameter); a patient environment parameter; and/or a system parameter. In some embodiments, a sensor or other functional element is configured to perform a diagnostic function (e.g. to gather data used to perform a diagnosis). In some embodiments, a functional element is configured to perform a therapeutic function (e.g. to deliver therapeutic energy and/or a therapeutic agent). In some embodiments, a functional element comprises one or more elements constructed and arranged to perform a function selected from the group consisting of: deliver energy; extract energy (e.g. to cool a component); deliver a drug or other agent; manipulate a system component or patient tissue; record or otherwise sense a parameter such as a patient physiologic parameter or a system parameter; and combinations of one or more of these. A functional element can comprise a fluid and/or a fluid delivery system. A functional element can comprise a reservoir, such as an expandable balloon or other fluid-maintaining reservoir. A “functional assembly” can comprise an assembly constructed and arranged to perform a function, such as a diagnostic and/or therapeutic function. A functional assembly can comprise an expandable assembly. A functional assembly can comprise one or more functional elements.

The term “transducer” where used herein is to be taken to include any component or combination of components that receives energy or any input and produces an output. For example, a transducer can include an electrode that receives electrical energy and distributes the electrical energy to tissue (e.g. based on the size of the electrode). In some configurations, a transducer converts an electrical signal into any output, such as: light (e.g. a transducer comprising a light emitting diode or light bulb), sound (e.g. a transducer comprising a piezo crystal configured to deliver ultrasound energy); pressure (e.g. an applied pressure or force); heat energy; cryogenic energy; chemical energy; mechanical energy (e.g. a transducer comprising a motor or a solenoid); magnetic energy; and/or a different electrical signal (e.g. different than the input signal to the transducer). Alternatively or additionally, a transducer can convert a physical quantity (e.g. variations in a physical quantity) into an electrical signal. A transducer can include any component that delivers energy and/or an agent to tissue, such as a transducer configured to deliver one or more of: electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising a piezo crystal); chemical energy; electromagnetic energy; magnetic energy; and combinations of one or more of these.

As used herein, the term “fluid” can refer to a liquid, gas, gel, or any flowable material, such as a material which can be propelled through a lumen and/or opening.

As used herein, the term “material” can refer to a single material, or a combination of two, three, four, or more materials.

As used herein, the term “lesion” comprises a segment of a blood vessel (e.g. an artery) that is in an undesired state. As used herein, lesion shall include a narrowing of a blood vessel (e.g. a stenosis), and/or a segment of a blood vessel, with or without narrowing, that includes a buildup of calcium, lipids, cholesterol, and/or other plaque.

It is appreciated that certain features of the present inventive concepts, which are, for clarity, described in the context of separate embodiments, may also be provided in combination in a single embodiment. Conversely, various features of the present inventive concepts which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the present inventive concepts have been simplified to focus on elements that are relevant for a clear understanding of the present inventive concepts, while eliminating, for purposes of clarity, other elements that those of ordinary skill in the art will appreciate may also comprise a portion of the present inventive concepts. However, because such elements are well known in the art, and because they do not necessarily facilitate a better understanding of the present inventive concepts, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

The present inventive concepts disclosed herein are directed to a system for producing a diagnostic result for a patient. The system includes one or more implantable monitoring devices, device 100 described herein, for implantation into a patient. Each device 100 can be configured as a cardiac monitoring device. Device 100 can include one or more devices that are implanted in the subcutaneous tissue of the patient and/or at another location under the skin of the patient. The diagnostic system can include a machine learning algorithm and/or other artificial intelligence algorithm (e.g. algorithm 166 and/or 266 described herein) that can be configured to monitor and/or reduce (e.g. prevent or at least reduce) arrhythmias and/or heart failure-related hospitalizations of the patient, such as via acquisition of patient physiologic information (also referred to as “vital signs” herein). In addition to arrhythmia monitoring, the diagnostic system can provide comprehensive cardiac and/or respiratory monitoring, including monitoring cardiac arrhythmias, heart valve function, heart failure, COPD, and voice changes (e.g. due to cardiac and/or respiratory illnesses and other respiratory diseases that can be benefited by tracking and/or trending changes in sounds). The diagnostic system of the present inventive concepts can be configured to monitor, determine, and/or assess (“monitor” herein) one or more patient physiologic parameters selected from the group consisting of: heart sound signatures; ECG data (e.g. via a single lead ECG); Electro-Mechanical Activation Time (EMAT); Systolic index of contractility (dP/dT); heart rate variability (HRV); patient activity; fluid impedance data; and combinations of these. The diagnostic system can monitor these parameters for a continuous period of time (e.g. continuously for up to 5 years). The diagnostic system can be configured to monitor one or more physiologic parameters of the patient at designated times throughout the day. The diagnostic system can be configured to perform machine learning (e.g. via data processing in a “cloud network”) to identify changes (e.g. subtle changes) from the acquired data (e.g. ECG, PCG, ICG, SCG, heart rate, other heart data, patient activity, body posture, body position, temperature, HRV, blood pressure, blood flow, and/or impedance such as fluid impedance).

The diagnostic systems of the present inventive concepts disclosed herein can include an electrocardiogram recording device configured to detect slow, fast, and/or irregular heartbeats, such as to calculate: heart rate; heart rate regularity (e.g. irregularity); heart rate variability; and/or other electrocardiogram-related analyses, including but not limited to, arrhythmia, dizziness, palpitations, chest pain, and/or shortness of breath. The diagnostic system can be configured to provide continuous and/or long-term monitoring of the patient, such as to aid in providing an accurate diagnosis. The diagnostic system can include a phonocardiogram to detect changes in heart sounds (e.g. S1, S2, S3, S4) and/or lung sounds. The diagnostic system can include an accelerometer (e.g. a three-axis accelerometer configured to sense vibrations in one, two, or three axes). The accelerometer can be configured to record multiple sets of data, simultaneously or sequentially, as described herein. The accelerometer can be configured to record acoustic data (e.g. heart sounds as described herein), such as when implantable device 100 does not include any standard microphone components for sound recording, such as when all sound recording is performed by one or more accelerometers of device 100. The diagnostic system can include one, two, or more temperature sensors, such as when the diagnostic system performs a trending analysis to monitor changes in body temperature such as changes that occur during resting, exercising, ovulations, and/or stress. The diagnostic system can include one, two, or more oxygen sensors configured to measure an oxygen level of a patient. The diagnostic system can include a device for monitoring blood pressure and/or blood flow, such as a device including one or more optical and/or acoustic sensor used to identify similar stressors of the patient. The diagnostic system can include a device for monitoring fluid impedance, such as a device including two embedded electrodes that are assigned to record electrocardiogram data. The diagnostic system can be configured to operate using voice activation and/or tactile activation.

The diagnostic system of the present inventive concepts can include one or more devices (e.g. one or more implantable devices and/or one or more external devices) that each include one or more sensors for collecting data that can be used by the diagnostic system to determine changes have occurred in patient vital signs, such as changes that occur over extended periods of time. Typical parameters monitored include but are not limited to: electrocardiogram; phonocardiogram; seismocardiography; temperature; patient activity level; blood pressure; blood flow; fluid impedance level (e.g. intra-thoracic fluid impedance level); oxygen sensor; and combinations of these. As heart failure progresses, the diagnostic system can detect strain in heart muscles and/or fluid changes, such as when these changes are associated with changes in S1, S2, S3, and/or S4 heart sound signature recordings. In order to provide an accurate and reliable assessment of heart failure or other patient medical condition status, the diagnostic system can perform continuous (e.g. and long-term) phonocardiogram monitoring combined with monitoring of one or more of: ECG; activity level; temperature; posture; body position; blood pressure; blood flow; and/or impedance (e.g. fluid impedance).

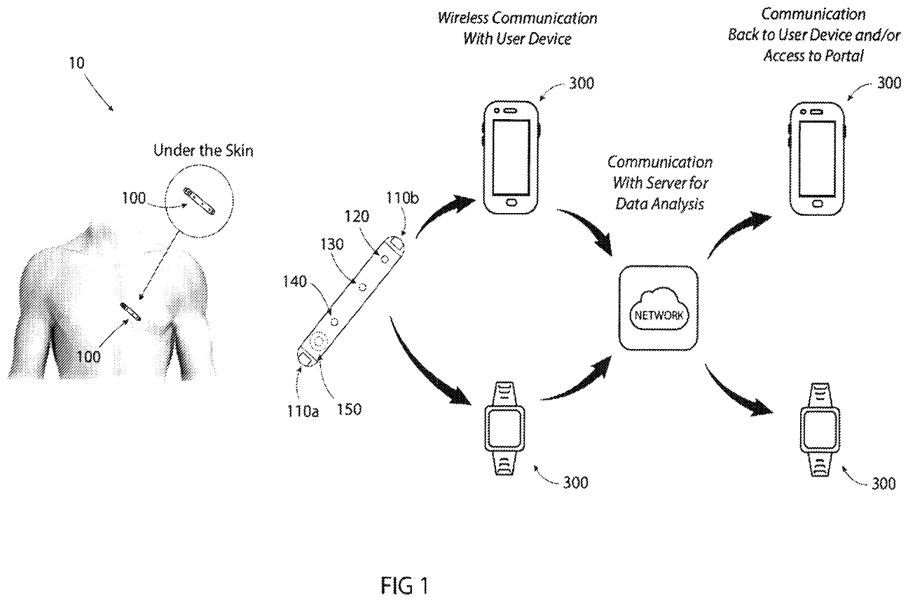

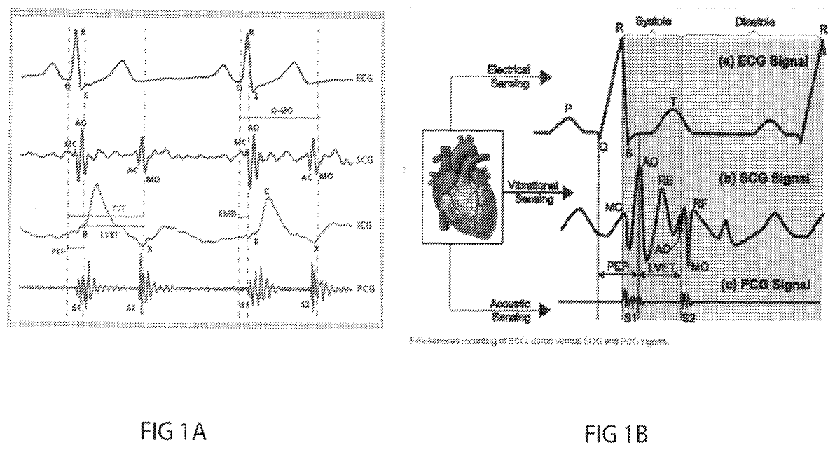

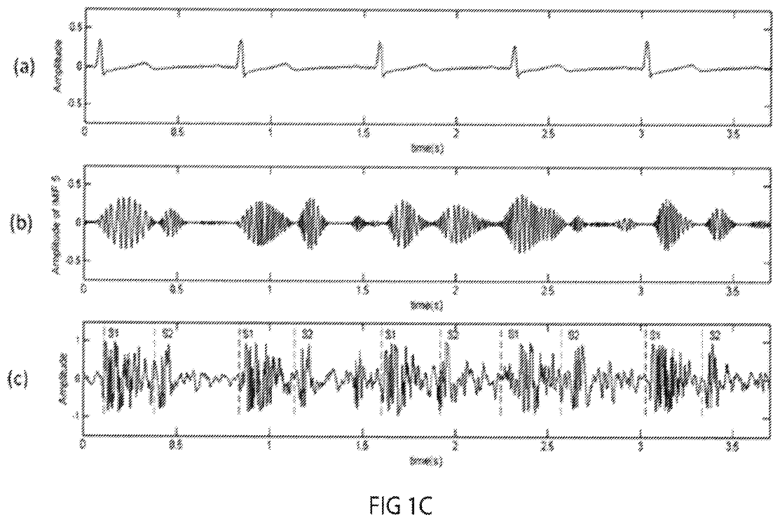

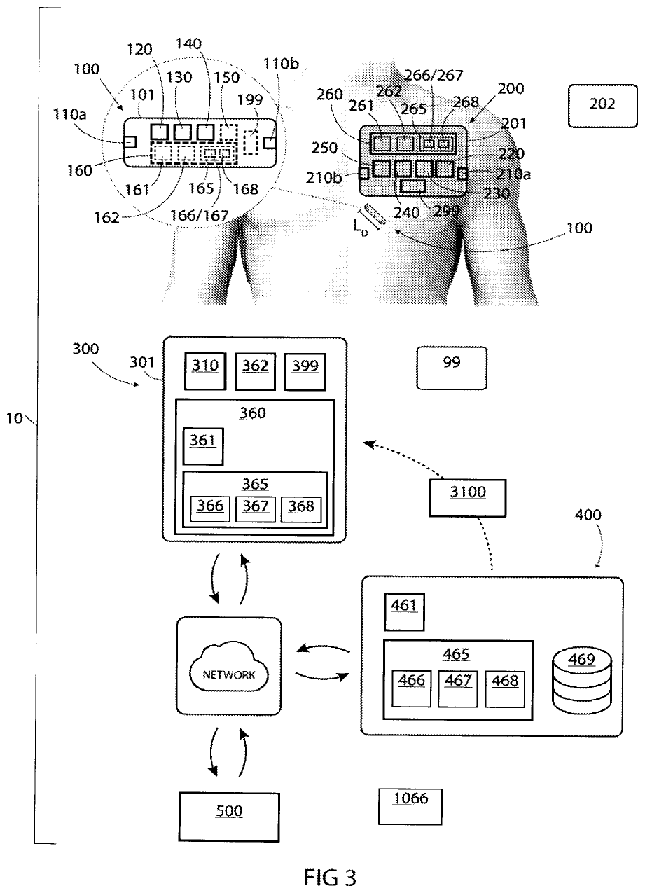

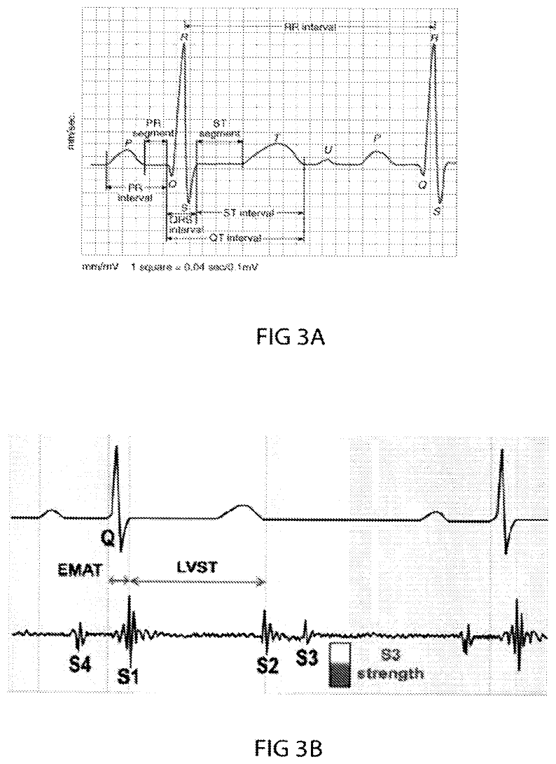

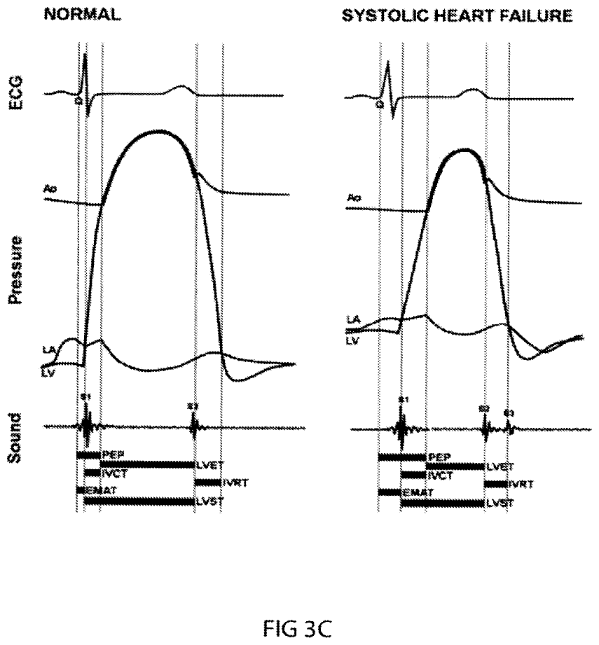

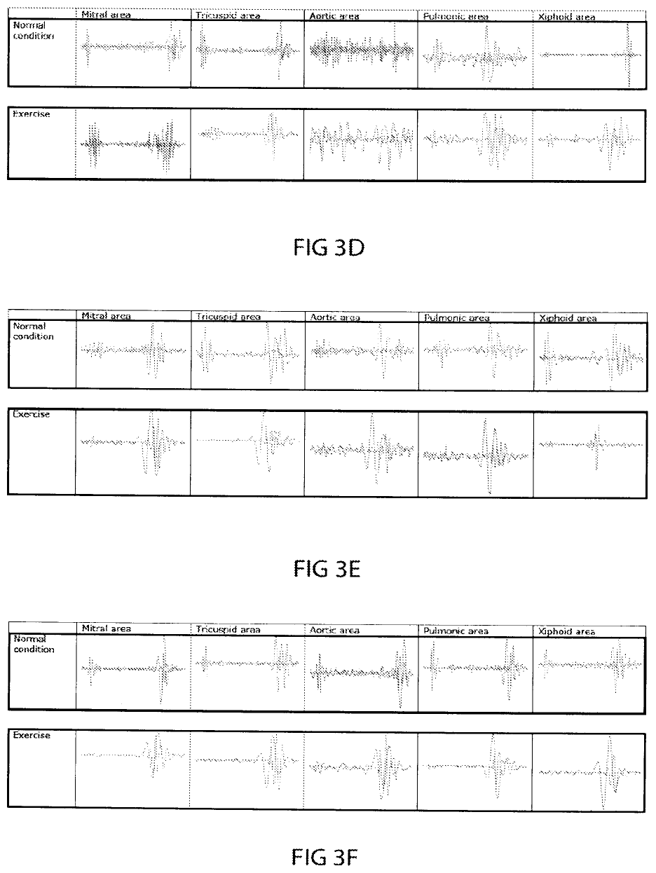

Referring now to FIG. 1, a schematic view of a patient diagnostic system including an implantable cardiac monitoring device in communication with various other system devices is illustrated, consistent with the present inventive concepts. System 10 can be configured to produce a diagnosis and/or prognosis for the patient, “diagnostic result” herein. The diagnostic result can include one, two or more diagnoses for the patient, and/or one, two, or more prognoses, for the patient. System 10 includes an implantable device, device 100 shown, which can be configured to communicate with one, two, or more other devices (e.g. implanted or external patient devices, or other devices) of system 10, such as a computer, tablet, smartphone, smartwatch, and the like. Device 100 can comprise one, two, or more implantable devices configured to perform cardiac monitoring, as described herein. Device 100 can be implanted into a patient, such as a subcutaneous implantation. Device 100 can comprise one, two, or more elements and/or other components similar to those included in conventional pacemakers and/or other implantable devices, including, but not limited to: a biocompatible housing; electrocardiogram (ECG) module (e.g. from sensors located proximate distal tips of the device); a microphone, or other type of acoustic capturing device (e.g. a device including an accelerometer, crystals, a ceramic, and/or an alloy), such as for providing a phonocardiogram (PCG), lung sounds, and/or pressure data; an accelerometer (e.g. a three-axis accelerometer or other sensor for monitoring and/or recording heart sounds, patient activity level, body posture and/or body position); temperature sensor; blood pressure sensor; blood flow sensor; impedance sensor, such as a fluid (electrical) impedance sensor; an embedded antenna, such as for communication with designated input devices (e.g. pacemaker programmer) and/or external devices (e.g. for communicating data); a battery; a computing module (e.g. a processing unit); and/or communications devices (e.g. Bluetooth or other wireless communication module). In some embodiments, system 10 is constructed and arranged similar to system 10 described herebelow in reference to FIG. 3.

Device 100 and/or another component of system 10 can comprise a sound capturing module (e.g. a sensor 120 described herein comprising an accelerometer, microphone, ceramic element, and/or crystal element) configured to record heart or other physiological sounds produced within the patient. The sound capturing module can comprise a component (e.g. an accelerometer) configured to sense physical vibrations and record those vibrations as digital data, such as to monitor heart sounds and/or blood pressure. In some embodiments, the sound capturing module can comprise a sensor (e.g. a sensor 120 comprising a vibrational sensor) configured to measure changes in the patient's voice, which can be indicative of fluid accumulation in the patient's lung and/or a change in the cardiac condition of the patient. In some embodiments, the sound capturing device records vibrations as an analog signal which is subsequently converted to a digital signal.

Device 100 and/or another component of system 10 can comprise a fluid impedance sensor (e.g. a sensor 120 comprising a fluid impedance sensor), such as a thoracic fluid impedance sensor configured to monitor (e.g. measure and/or analyze) fluid accumulation in the lungs, such as a monitoring performed using prior trans-thoracic and intra-thoracic fluid impedance measurements. Measuring and/or analyzing fluid accumulation in the lungs performed by system 10 can provide additional criteria for monitoring a heart failure status of the patient (e.g. heart failure diagnosis and/or prognosis of the patient). Device 100 can comprise at least two electrodes (e.g. electrodes 110 described herein) configured to monitor electrical resistance between two points within the patient's anatomy. For example, if the impedance measured between the two electrodes is high (e.g. a high resistance), it can be determined by system 10 that the lungs (e.g. lung tissue and/or surrounding tissue) are dry and/or in good condition (e.g. indicating an acceptable level of thoracic fluid). However, if the impedance measured between the two electrodes is low (e.g. a low resistance), it can be determined by system 10 that the lungs (e.g. lung tissue and/or surrounding tissue) are wet and/or in poor condition (e.g. indicating an increased level of thoracic fluid). In some embodiments, system 10 includes an additional sensor for measuring fluid level (e.g. a sensor 120 configured to measure fluid level of a lung), such as an external patch which performs electrical recordings that in effect widen the vector and the surface area as compared to the recordings made by the sensor(s) of device 100 alone.

In some embodiments, device 100 comprises a volume of no more than 2.5 cm³, such as a volume of approximately 1.5 cm³. In some embodiments, device 100 comprises dimensions of less than 60 mm by 16 mm by 7 mm, such as when comprising dimensions of approximately 44 mm by 7 mm by 4 mm. In some embodiments, device 100 comprises a mass of less than 3.5 g, such as a mass of approximately 2 gm. In some embodiments, device 100 comprises at least two electrodes (e.g. two electrodes 110) that are positioned a distance of approximately 44 mm apart.

Device 100 can be configured to communicate (e.g. transmit data) with at least one patient device and/or other device of system 10, such as communication performed via bidirectional low energy communication methods. Device 100 can be configured to wirelessly communicate with at least one other device of system 10, such as via Bluetooth low energy transmission (e.g. Wi-Fi, NFC, RF). In some embodiments, device 100 is configured to communicate with at least one other system 10 device at least every 12 hours. In some embodiments, the frequency at which device 100 communicates with at least one system 10 device can be adjusted (e.g. programmed), such as by a clinician of the patient. If a communication link cannot be established between device 100 and a separate device of system 10 (e.g. no connection is available for a period of at least 1 hour, at least 3 hours, and/or at least 6 hours), system 10 can be configured to enter an alert state, such as a state in which system 10 contacts (e.g. via an email or phone call) a healthcare provider of the patient and/or a family member of the patient and provides information related to the issue. System 10 can be configured to allow a user, such as the patient, a family member of the patient, and/or a healthcare provider of the patient, to transfer data between two, three, or more devices of system 10, such as to transfer data from device 100 to a separate device of system 10 that is configured to analyze the transferred data.

System 10 can be configured to initiate a data recording session (e.g. a session in which device 100 and/or another device of system 10 begins monitoring a set of one or more patient physiologic parameters) when a particular patient condition is detected (e.g. via a patient parameter already being monitored exceeding a threshold), such as a condition in which the patient's heart rate is less than or equal to 50 bpm. System 10 can be configured to initiate a recording session when the patient's heart rate is greater than or equal to 90 bpm. In some embodiments, the minimum and/or maximum patient heart rate that causes system 10 to initiate a recording session can be adjusted (e.g. programmed) by a clinician.

System 10 can be configured to implement R to R wave (QRS wave of ECG) sensing capabilities for slow, regular, irregular, and/or fast heart rates. System 10 can be configured to implement R to R sensing capabilities for regularity and/or irregularity in heart rates (e.g. atrial fibrillation, ventricular tachycardia, and/or other arrhythmias).

System 10 can be configured to determine a heart rate and activity calculation to perform a heart rate variability (HRV) assessment.

System 10 can be configured to collect ECG data and/or determine one or more output values (e.g. calculated values) that are based on the ECG data. In some embodiments, device 100 and/or another component of system 10 is configured to listen to and/or otherwise monitor (“monitor” herein) and/or record every heartbeat of the patient. In some embodiments, system 10 is configured to monitor to monitor and/or record heartbeats according to various timing configurations. As an example, device 100 can be configured to monitor and/or record heartbeats for a 1 minute time period repeated every 15 minutes. As another example, device 100 can be configured to monitor and/or record heartbeats for a 20 second time period repeated every 5 minutes. In some embodiments, device 100 is configured to record data (e.g. heartbeats) on command, such as by a manual activation by a user (e.g. the patient or a clinician). Alternatively or additionally, device 100 can be configured to record data (e.g. heartbeats) based on programmed parameters (e.g. as entered by a user).

System 10 can be configured to collect phonocardiogram (PCG) data (also referred to as heart sound data) and/or determine one or more measurements based on PCG data. In some embodiments, device 100 is configured to monitor every heartbeat of the patient. In some embodiments, device 100 is configured to monitor and/or record (either or both, “monitor” or “record” herein) every heartbeat of the patient. In some embodiments, device 100 is configured to monitor and/or record heartbeats according to various timing configurations, such as automatic and/or manual, pre-programmed, and/or on demand timing configurations of recordings, as described herein. In some embodiments, device 100 is configured to record data (e.g. PCG data) on command, such as by a manual activation by a user (e.g. the patient, a family member, and/or a clinician). Alternatively or additionally, device 100 can be configured to record data (e.g. PCG data) based on programmed parameters (e.g. as entered by a user). As an example, device 100 can be configured to monitor and/or record heartbeats for a 1 minute time period repeated every 15 minutes. As another example, device 100 can be configured to monitor and/or record heartbeats for a 20 second time period repeated every 5 minutes.

As described herein, it may not be necessary for system 10 to be configured to monitor and/or record every heart sound of the patient. In some embodiments, system 10 (e.g. device 100) is configured to monitor and/or record heart sounds for a 1 minute time period repeated every 15 minutes. In other embodiments, device 100 is configured to monitor and/or record heart sounds for a 20 second time period repeated every 5 minutes. In yet another embodiment, device 100 is configured to automatically monitor and/or record heart sounds when the patient's heart rate increases to a pre-determined parameter. Exemplary data (ECG and sound signatures) collected by device 100 is shown in FIGS. 1A-C. By utilizing multiple sensors, system 10 (e.g. device 100) can be configured to record electrical, mechanical, and/or physiological biomarkers, such as to increase system 10 accuracy (e.g. increase specificity and/or sensitivity). System 10 can include a machine learning algorithm and/or other artificial intelligence algorithm (e.g. algorithm 166 and/or 266 described herein) that is configured to monitor subtle changes in collected data over long periods of time, such as to assess improvements and/or regression of cardiac performance and/or other health condition of the patient. For example, in heart failure, ECG data may not indicate significant changes in electrical activation, however, heart sounds (PCG) can indicate notable changes that relate to a heart failure condition. System 10 can be configured to provide high accuracy diagnoses when both ECG and PCG are used in conjunction to produce a diagnostic result of the present inventive concepts. Furthermore, system 10 can include data from an accelerometer (e.g. a three-axis accelerometer) in order to increase accuracy, such as by monitoring variations of heart sounds based on body position over an extended period of time, allowing the clinician and/or system 10 (e.g. via the machine learning algorithm) to discern the origins of the specifics of the heart sounds. The accelerometer can be configured to record pressure, which can then be converted into a sound signature. In some embodiments, the accelerometer can comprise a three-axis accelerometer, such that the direction and/or the origin of the recorded sounds can be determined via signals recorded relative to the three axes (e.g. to precisely locate the heart sounds such as to remove or otherwise account for outside noise). System 10 can overcome shortcomings of “over-sensing” and “under-sensing” of ECG by employing additional sensors to aid in ECG monitoring. As an example, if under-sensing, the ECG may be indicative of asystole (zero cardiac activity), while recorded PCG data may comprise normal heart sounds along with normal activity level, such that analyses performed by system 10 using this collective data would result in system 10 indicating the patient is not in an adverse state. System 10 can monitor and analyze data from these, and other multiple parameter sets, to reduce false positives and false negatives.

System 10 can be configured to monitor and/or record heart sounds according to various timing configurations, such as when recording is based on programmed timing parameters and/or manually as initiated by a user. As an example, system 10 can be configured to monitor and/or record heart sounds for a particular time period, repeated at a particular frequency, such as a 1 minute period repeated every 15 minutes. As another example, system 10 can be configured to monitor and/or record heart sounds for a 20 second time period repeated every 5 minutes. As yet another example, system 10 can be configured to monitor and/or record heart sounds continuously. In addition to acquiring heart sounds, system 10 can be configured to acquire ECG data, temperature data, blood pressure data, blood flow data, patient activity level data, heart rate data, and/or fluid impedance level data in similar timing configurations. In some embodiments, system 10 can be configured to acquire heart sound data and ECG data, as well as two, three, or more of: temperature data; blood pressure data; blood flow data; patient activity level data; heart rate data; and/or fluid impedance level data, and produce a diagnostic result based on that data set.

Implantation of device 100 can be performed during a brief (e.g. between 1 and 5 minutes) procedure in an office, outpatient clinic, or hospital setting. Device 100 can be implanted under a portion of the patient's skin located in the pectoral region (e.g. the left pectoral region, between rib number 4 through 6). Device 100, or a portion of device 100, can be implanted under a portion of the patient's skin located on the back of the torso, such as to enhance the ability of device 100 to identify lung sounds. Device 100 can be implanted under a portion of the patient's skin located proximate a joint (e.g. knee, elbow), such as to enhance the ability of device 100 to identify tendon strain sounds (e.g. when an algorithm of system 10 uses at least tendon strain sound data to produce a diagnostic result). Device 100 can be implanted under a portion of the patient's skin located proximate an artery and/or vein (e.g. carotid artery, and/or a blood vessel of the wrist, back of knee, groin, armpit, or other vascular rich region), such as to enhance the ability of device 100 to auscultate arterial and/or venous flow, such as to monitor blood pressure and/or other vital signs (e.g. and use the collected data to produce a diagnostic result).

A patient device of system 10 can be configured as a communication conduit between device 100 and a computer network, such as the cloud network.

System 10 can be configured as a reporting tool to one or more clinicians and/or other users of system 10.

System 10 can be configured to provide accurate data in a timely manner, such as when system 10 is configured to bypass the otherwise cumbersome communication chain at a hospital facility level.

System 10 can be configured to communicate with a cloud-based machine learning algorithm and/or other artificial intelligence algorithm (e.g. an algorithm of system 10, such as algorithm 166 and/or 266 described herein), such as via at least one device of system 10. The machine learning algorithm can be configured to monitor all patient vital signs, monitor arrhythmias, and/or prevent or otherwise reduce heart failure admissions.

A machine learning algorithm and/or other artificial intelligence algorithm (“machine learning algorithm” herein) of system 10 can be configured to perform a backend analysis of trending data and/or pattern recognition over days (e.g. at least 2 days), months (e.g. at least 2 months), and/or years (e.g. at least 2 years) of one or more patient vital signs.

A machine learning algorithm of system 10 can be configured to gather data (e.g. ECG, PCG, ICG, SCG, heart rate, other heart data, patient activity, body posture, body position, temperature, HRV, blood pressure, blood flow, and/or impedance such as fluid impedance) recorded by system 10 (e.g. recorded by at least device 100) such as to analyze data points from each heart sound recording and/or measurement, such as heart sound signatures S1, S2, S3, S4. Heart sound signatures S1, S2, S3, S4 can exhibit subtle changes throughout the progression of heart failure disease that can be detected (e.g. recorded and identified in a diagnostic result) by system 10. Notably, changes in S1, S2, S3, and S4 can be correlated to early signs of a declining heart condition of the patient. By collecting multiple aspects of heart sound signatures (e.g. along with ECG and/or other patient parameters), system 10 (e.g. via an included machine learning algorithm) can provide a diagnostic result comprising several heart ailment models.

A machine learning algorithm of system 10 can be configured to identify and/or analyze changes (e.g. subtle changes) in cardiac filling and ejection sounds (e.g. with 100,000 to 100,000,000 samples of high-fidelity heart sound data to compare with, such as previous patient population data including billions of data points within each heart sound). In general, a patient's heart beats approximately 100,000 time a day. System 10 can analyze heartbeat data that are recorded over days, weeks, months and/or years, such as to identify subtle changes (e.g. using machine learning and trending analysis).

A machine learning algorithm of system 10 can be configured to identify and/or analyze changes (e.g. subtle changes) in HRV, such as to assess the patient's cardiac and/or overall health condition. For example, low HRV can be used by system 10 to determine a declining heart health.

A machine learning algorithm of system 10 can be configured to identify and/or analyze changes (e.g. subtle changes) in patient temperature. For example, system 10 can use machine learning to analyze patient temperature changes that can be indicative of various events, including but not limited to: performance, ovulation cycle, fatigue, stress, illness, and/or other temperature-related patient medical conditions.

A machine learning algorithm of system 10 can be configured to identify and/or analyze changes (e.g. subtle changes) in patient blood pressure. For example, system 10 can use machine learning to analyze patient blood pressure changes that can be indicative of various events, including but not limited to: pregnancy, fatigue, stress, illness, and/or other blood pressure-related patient medical conditions.

A machine learning algorithm of system 10 can be configured to identify and/or analyze trending changes (e.g. subtle changes) in fluid level and/or mechanical strain in the patient's heart and/or lungs.

System 10 can be configured to utilize a machine learning algorithm according to the following protocol:
STEP 1: System 10 (e.g. device 100) acquires data from the patient, such as: ECG, PCG, ICG, SCG, heart rate, other heart data, patient activity, body posture, body position, temperature, HRV, blood pressure, blood flow, and/or impedance such as fluid impedance. Data can be gathered from various locations within the patient, and/or on the skin or otherwise proximate the patient. In some embodiments, data is gathered from areas proximate the wrist, proximate the ear (e.g. behind the ear), and/or proximate one, two, or more blood vessels.
STEP 2: A system 10 device (e.g. device 100) communicates and transfers the acquired data to at least one other device of system 10, such as a smartphone, smart watch, smart glasses, smart headphones, external patches, and/or other system 10 device external to the patient. In some embodiments, at least device 100 wirelessly communicates with the at least one external device of system 10. In some embodiments, the external device is also configured as a display module for access to the data by the clinician or other user of system 10.
STEP 3: The external device communicates and transfers the data to a patient portal of system 10 in a cloud network. As described herein, the patient portal can comprise a machine learning algorithm (e.g. algorithm 166 and/or 266 described herein) that is configured to analyze and/or manipulate the data.
STEP 4: The machine learning algorithm evaluates and compares the data to patient data that was previously collected (e.g. from a previous time period of hours, days, weeks, months, and/or years), such that the algorithm compares and contrasts the current data with previously collected data.
STEP 5: The machine learning algorithm can smooth outlying data points, such as data points that represent excessive noise and/or other anomalies. In some embodiments, the outlying data points are noted and monitored for trending data. Outlying data may show patterns after an extended period of time; thus, those data can be also used for further data analysis.
STEP 6: The patient portal displays or otherwise communicates the analyzed data to a clinician or other user. In some embodiments, the patient portal allows the user to visualize trending changes in heart signatures. In some embodiments, the patient portal communicates the analyzed data to the user via a personal device, such as a smartphone or smart watch, and thereby enabling the clinician or other user to assess the patient's condition.

System 10 can comprise one or more devices that are configured to be implanted under the patient's skin (e.g. one or more implantable devices 100, as described herein). System 10 can further comprise one or more devices that are configured to be positioned on the patient's skin and/or otherwise be located proximate the patient during their use (e.g. one or more external devices 200, as described herein). Each of the implanted and external devices can include one or more sensors configured to record physiologic information of the patient, and/or information related to the patient's environment (e.g. produce a signal representing those parameters). In some embodiments, sensors of one or more implantable devices (e.g. sensors 110, 120, 130, 140, 150 and/or 190 described herein) are configured to accurately record changes in physiologic parameters that occur within a time period of no more than 10 milliseconds, 7 milliseconds, 4 milliseconds and/or 2 milliseconds. In some embodiments, one or more of these implanted sensors are further configured to record changes in physiologic parameters representing tissue motion that is less that or equal to 5 mm, 3 mm, 1.5 mm, and/or 0.5 mm (e.g. the associated implanted sensors have a specificity and accuracy to reliably detect motion of that small magnitude).

System 10 can comprise one or more implantable devices 100 that each comprise an accelerometer 150 representing a 3-axis accelerometer. Each accelerometer 150 can be configured to produce a signal that represents vibrations generated from the heart of the patient, such that an algorithm of system 10 (e.g. algorithm 166, 266, 366, 466, and/or 1066 described herein) can convert the vibrations recorded by accelerometer 150 into sound signatures of the patient (e.g. as described herein). In some embodiments, one or more of the algorithms of system 10 analyzes the sound signature data, as well as one or more of (e.g. all of) body activity data, body posture data, and/or body orientation data (e.g. each as recorded by an implanted and/or external sensor), to produce a diagnosis result related to a medical condition of a patient, as described herein.

System 10 can comprise at least one sensor configured to produce a signal related to heart sounds (e.g. one or more accelerometers 150) and at least one sensor configured to produce a signal related to ECG of the patient (e.g. one or more of electrodes 110 and/or 210). These sensors can be configured to accurately record changes in the associated physiologic parameters that occur within a time period of no more than 10 milliseconds, 7 milliseconds, 4 milliseconds and/or 2 milliseconds. In some embodiments, an algorithm of system 10 (e.g. algorithm 166, 266, 366, 466, and/or 1066 described herein) determines at least the electromechanical activation time and the left ventricular systolic time based on at least the heart sounds and ECG.

In some embodiments, system 10 comprises one or more implantable devices 100 that each comprise a power supply 162 that is configured to provide sufficient energy to the associated implantable device 100 to support use over at least a two-year period. In these embodiments, system 10 can be configured to: perform a trending analysis of one or more recorded patient physiologic parameters, and/or perform a trending analysis of a diagnostic result that is based on the recordings of one or more patient parameters. The one or more trending analyses can represent a trend that occurs over a time period of between one and seven days, between one and six months, and/or between one and two years.

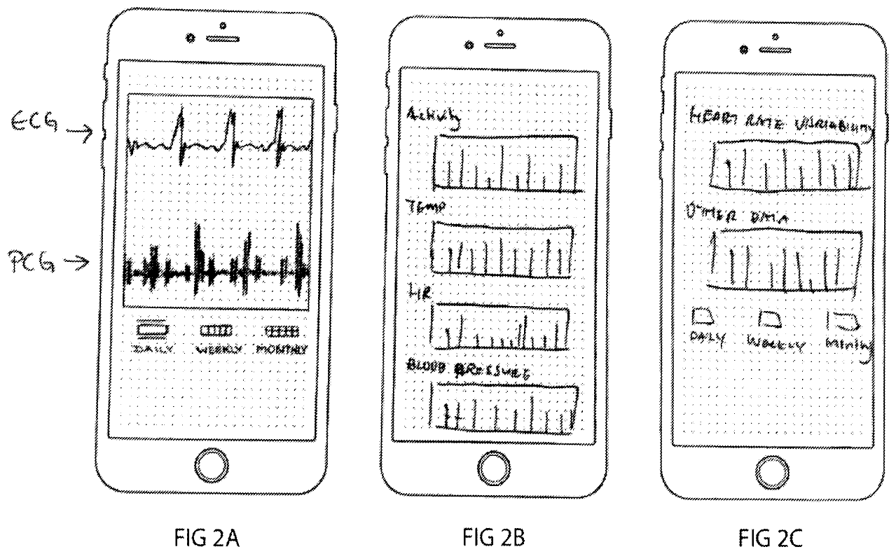

Referring now to FIGS. 2A-C, external devices displaying various physiological recordings based on data collected by at least an implantable cardiac monitor are illustrated, consistent with the present inventive concepts. Referring specifically to FIG. 2A, the external device can display ECG and PCG graphs based on data collected by at least device 100, and the data can be displayed related to time periods of a day, a week, and/or a month. Referring specifically to FIG. 2B, the external device can display patient activity, temperature, heart rate, blood pressure, and/or blood flow graphs based on data collected by at least device 100, and the data can be displayed related to time periods of a day, a week, and/or a month. Referring specifically to FIG. 2C, the external device can display HRV and D-dimer data graphs based on data collected by at least device 100, and the data can be displayed related to time periods of a day, a week, and/or a month.

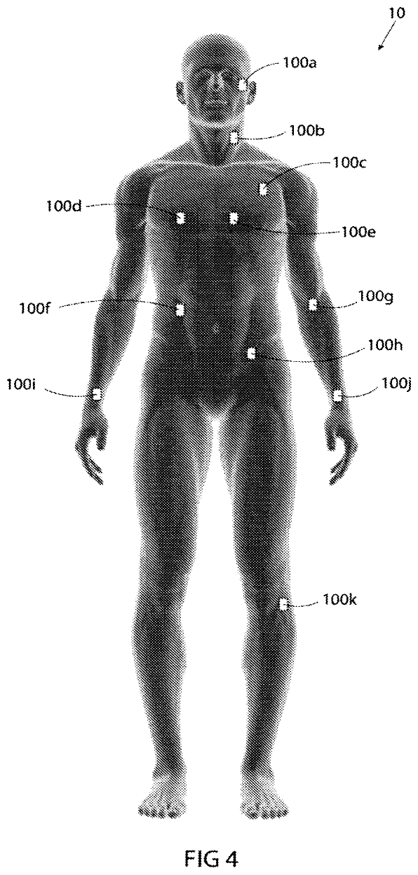

Referring now to FIG. 3, a schematic view of a patient diagnostic system including an implantable device is illustrated, consistent with the present inventive concepts. System 10 can be configured to produce a diagnosis and/or prognosis for the patient, “diagnostic result” herein. The diagnostic result can include one, two or more diagnoses for the patient, and/or one, two, or more prognoses, for the patient. System 10 includes one, two, or more implantable devices, implantable device 100 shown. Implantable device 100 comprises one, two, or more devices that are configured to be implanted under the skin of a patient (e.g. implantable device 100 can be implanted into subcutaneous tissue) and collect patient data (e.g. patient physiologic data) from one or more sensors positioned under the skin of the patient. In some embodiments, implantable device 100 comprises multiple devices 100, such as is described in reference to FIG. 4 herein. In these embodiments, a first device 100 can be implanted in a first location and a second device 100 can be implanted in a second location, such as a location relatively remote from the first location, such as to enhance (e.g. make more accurate and/or more specific) the diagnostic result produced by system 10 based on the data that is recorded from the two separate (remote) locations. For example, a first device 100a can be implanted in the chest of the patient (e.g. the upper left or right chest area), and a second device 100b can be implanted in the abdomen of the patient (e.g. the lower left or lower right abdominal area). System 10 can further include one or more patient devices maintained external to the patient, external device 200 shown, such as one or more devices that are worn by the patient (e.g. adhered to the skin of the patient or worn in a pocket of a garment, as described herein), and/or are maintained in relatively close proximity to the patient, and collect patient data (e.g. patient physiologic data) and/or patient environment data (e.g. temperature, humidity, light intensity, and/or barometric pressure of the current environment of the patient) from one or more sensors positioned external to the patient. System 10 can further include one or more other external devices, user device 300 shown, such as one or more devices that communicate with either or both of implantable device 100 and/or external device 200. In some embodiments, user device 300 also communicates with one or more remote systems, such as via the Internet or other wired or wireless network. For example, user device 300 can communicate with a remote data storage and/or processing device, server 400. Additionally or alternatively, device 300 can communicate with one or more other user devices 300, such as other user devices 300 of other patients, as described herein. System 10 can further include one or more clinician devices 500, and each of these can be configured to communicate with server 400 and/or one or more user devices 300. System 10 comprises a diagnostic system configured to diagnose and/or prognose (“diagnose” herein) a patient medical condition (e.g. a patient disease and/or disorder), such as to produce a diagnostic result (e.g. a diagnostic result that is provided to a clinician of the patient). In some embodiments, system 10 can be further configured to treat one or more medical conditions of the patient, such as when treating a medical condition being diagnosed by system 10 (e.g. and provided in the form of a “diagnostic result”).

Implantable device 100 comprises one or more housings, housing 101 shown, surrounding the various components of implantable device 100. One or more components of implantable device 100 can extend through at least a portion of housing 101, such as to interact with surrounding patient tissue, such as to measure one or more parameters of the surrounding tissue, as described herein. Housing 101 can comprise a length L_{D}. The length L_{D} can comprise a length of less than 60 mm, such as less than 50 mm, such as less than 40 mm, such as less than 30 mm. Housing 101 can comprise a cross-sectional (e.g. perpendicular to length L_{D}) shape comprising a shape approximating a rectangle or an ellipse. In some embodiments, the major axis of the cross-sectional shape of housing 101 comprises a length of less than 10 mm, such as approximately 7 mm. Additionally or alternatively, the minor axis of the cross-sectional shape of housing 101 can comprise a length of less than 5 mm, such as approximately 4 mm. In some embodiments, housing 101 can comprise a volume of less than 2 cm³, such as approximately 1.5 cm³. Implantable device 100 can comprise a mass of less than 5 gm, such as approximately 2 gm. Housing 101 can comprise a biocompatible housing, such as a housing comprising one or more biocompatible materials. Implantable device 100 (e.g. housing 101) can include one or more coatings configured to enhance adherence, or reduce adherence, to the surrounding tissue. Implantable device 100 (e.g. housing 101) can be treated with fractional surfacing, such as to increase the exposed surface area. Implantable device 100 (e.g. housing 101) can be impregnated and/or coated with an agent (e.g. a steroid, anti-inflammatory, anti-thrombogenic, and/or other pharmaceutical), such as to reduce tissue inflammation and/or to decrease the likelihood of infection. Implantable device 100 can include various shapes to increase the surface area without significantly changing the shape or the overall length or width. In some embodiments, implantable device 100 includes one or more components configured to allow voice-based activation and/or tactile-based activation.

Implantable device 100 can comprise one or more electrical components, such as first electrode 110a and second electrode 110b shown (collectively or singularly electrode 110 herein). Electrodes 110 can be positioned on opposite ends of housing 101, such that they are positioned approximately a distance equal to L_{D} from each other. In some embodiments, sensor 120, described herein, comprises one, two, three, or more electrodes 110. In some embodiments, at least a portion of housing 101 is configured as an electrode 110 (e.g. a metallic or other conductive portion of housing 101, such as a portion that is configured as a cathode). At least a portion of one or more electrodes 110 can be positioned outside of housing 101, such that each electrode 110 is in contact with the tissue and/or body fluids of the patient. For example, a surface of an electrode 110 can be coplanar with a portion of housing 101 surrounding the electrode 110 surface, such that the outer surface of implantable device 100 is relatively smooth at the electrode 110 location (e.g. electrode 110 does not protrude beyond an outer surface of housing 101). In some embodiments, electrode 110 can comprise an effective surface area (e.g. a surface area exposed to the tissue and/or fluids of the patient) of at least 2 mm². In some embodiments, the exposed surfaces of electrodes 110a and 110b can be facing opposite directions (e.g. the exposed surface of first electrode 110a is facing away from the center of implantable device 100 at a first end of device 100, and the exposed surface of second electrode 110b is positioned at a second end of device 100 facing the opposite direction). Alternatively, the exposed surfaces of electrodes 110 can be relatively coplanar, for example, when each electrode 110 exposed surface is positioned on the same side of housing 101, at opposite ends of device 100. In some embodiments, first electrode 110a and/or second electrode 110b each comprise multiple electrodes, such as two, three, or more electrodes. In some embodiments, multiple first electrodes 110a are positioned circumferentially about a first end of device 100, and multiple second electrodes 110b are positioned circumferentially about a second end of device 100. In some embodiments, electrodes 110 are utilized by system 10 to record ECG signals of the patient, as described herein. In some embodiments, additional electrodes 110 can be included to enhance the vector dynamic between two different patient locations and/or multiple patient locations. In some embodiments, the electrodes 110 are incorporated into housing 101 to result in a smooth surface, as described herein.

Implantable device 100 can comprise one or more audio recording devices and/or other acoustic sensors, sensor 120. Sensor 120 can comprise a microphone, an accelerometer (e.g. an accelerometer configured to record sound signals), and/or other acoustic sensor component. Alternatively or additionally, sensor 120 can comprise a sensor selected from the group consisting of: a pressure sensor; a blood pressure sensor; a blood flow sensor; a temperature sensor; a strain gain; a blood glucose sensor; a blood gas sensor; a respiration sensor; an EKG sensor (e.g. one, two, or more electrodes); a neuronal firing sensor (e.g. a sensor configured to record neuronal spikes, local field potential signals, and/or EEG signals); a flow sensor (e.g. a blood flow sensor); and combinations of one, two, or more of these. Sensor 120 can comprise one, two, or more electrodes (e.g. at least a pair of electrodes), such as electrodes 110 described herein. Sensor 120 can comprise one, two, three, or more sensors configured to record one, two, three, or more physiologic parameters of the patient. In some embodiments, sensor 120 comprises one or more sensors configured to record two, three, or four of: heart sounds, patient movement, EEG, blood flow, and/or patient temperature, such that the diagnostic result produced by system 10 can be based on each of the recorded signals. In these embodiments, the recorded data can be received by one or more external devices 200, such that the one or more external devices 200 can analyze the received data.

In some embodiments, sensor 120 comprises at least an accelerometer and at least one electrode, and the diagnostic result is based on data recorded by the at least an accelerometer and at least one electrode. In some embodiments, sensor 120 comprises at least a 3-axis accelerometer, as described herein, such that device 100 can, via the accelerometer, both record heart sounds and determine an origin of the recorded heart sounds.

At least a portion of sensor 120 can be positioned outside of housing 101 (e.g. relatively coplanar with the surface of housing 101), such that at least a portion of sensor 120 is in contact with tissue and/or fluids of the patient. Alternatively or additionally, sensor 120 can be positioned within housing 101, and configured to record sounds (e.g. heart or lung sounds) that are present within housing 101. In some embodiments, housing 101 comprises a material with a high acoustic transparency, such that sound can penetrate housing 101 and be recorded by an internally-located sensor 120. In some embodiments, housing 101 comprises one or more ports (not shown) configured to allow sound to at least partially penetrate housing 101. For example, housing 101 can comprise one or more ports (not shown) comprising a material (e.g. a flexible membrane material) configured to allow sound to pass through housing 101 while preventing fluid or other body contaminants from entering housing 101. Sensor 120 can comprise multiple acoustic sensors (e.g. multiple microphones, accelerometers, and/or other acoustic sensors), such as at least two acoustic sensors positioned at opposite ends and/or on opposite sides of implantable device 100. In some embodiments, sensor 120 is used by system 10 to record a PCG and/or lung sounds, as described herein. In some embodiments, sensor 120 utilizes device 100 as a source of acoustic amplification (e.g. avoiding sensor 120 from protruding from housing 101 into the patient).

Implantable device 100 can comprise one or more temperature sensors, temperature sensor 130 shown. At least a portion of temperature sensor 130 can be positioned outside of housing 101 (e.g. a temperature sensing portion that is relatively coplanar with the surface of housing 101), such that at least a portion of temperature sensor 130 is in contact with the tissue and/or fluids of the patient. Alternatively or additionally, temperature sensor 130 can be positioned within housing 101, and configured to record the temperature within housing 101 (e.g. within the space surrounded by housing 101). In some embodiments, housing 101 comprises a thermally conductive material, such that the temperature inside of housing 101 approximates the temperature of the tissue surrounding housing 101. In some embodiments, temperature sensor 130 comprises one or more temperature sensors that are incorporated within the wall thickness of housing 101 (avoiding a sensor 130 from protruding from housing 101 into the patient).

Implantable device 100 can comprise one or more pressure-sensitive transducers, pressure sensor 140 shown. At least a portion of pressure sensor 140 can be positioned outside of housing 101 (e.g. relatively coplanar with the surface of housing 101), such that at least a portion of pressure sensor 140 is in contact with the tissue and/or fluids of the patient. Alternatively or additionally, pressure sensor 140 can be positioned within housing 101 (e.g. within the space surrounded by housing 101), for example, when housing 101 comprises a port (not shown) configured to allow pressure changes within the tissue of the patient to be recognized by pressure sensor 140 from within housing 101. In some embodiments, pressure sensor 140 is utilized by system 10 to record the blood pressure and/or blood flow of a patient, as described herein. In some embodiments, pressure sensor 140 is incorporated within the wall of housing 101 (e.g. to avoid sensor 140 from protruding from housing 101 into the patient). In some embodiments, a single sensor (e.g. a single component) comprises both sensor 120 and pressure sensor 140 (e.g. a crystal and/or ceramic sensor that both records sound waves and measures pressure).

Implantable device 100 can comprise one or more accelerometers, accelerometer 150 shown. In some embodiments, sensor 120 comprises accelerometer 150. Accelerometer 150 can comprise one or more one-axis, two-axis, and/or three-axis accelerometers. In some embodiments, accelerometer 150 comprises two or more accelerometers, such as two, three-axis accelerometers positioned such that the axis of each of the multiple accelerometers is positioned in a unique orientation (e.g. providing at least six-axis data). In some embodiments, accelerometer 150 is utilized by system 10 to monitor heart sounds of the patient, the activity level of the patient, body position of the patient, and/or body posture of the patient. In some embodiments, accelerometer 150 comprises one, two, or more accelerometers configured to record at least two of: heart sounds of the patient, the activity level of the patient, body position of the patient, and/or body posture of the patient. In some embodiments, accelerometer 150 comprises, one, two, or more accelerometers configured to record at least three of: heart sounds of the patient, the activity level of the patient, body position, and/or body posture of the patient. In some embodiments, accelerometer 150 comprises, one, two, or more accelerometers configured to record all four of: heart sounds of the patient, the activity level of the patient, body position, and/or body posture of the patient.

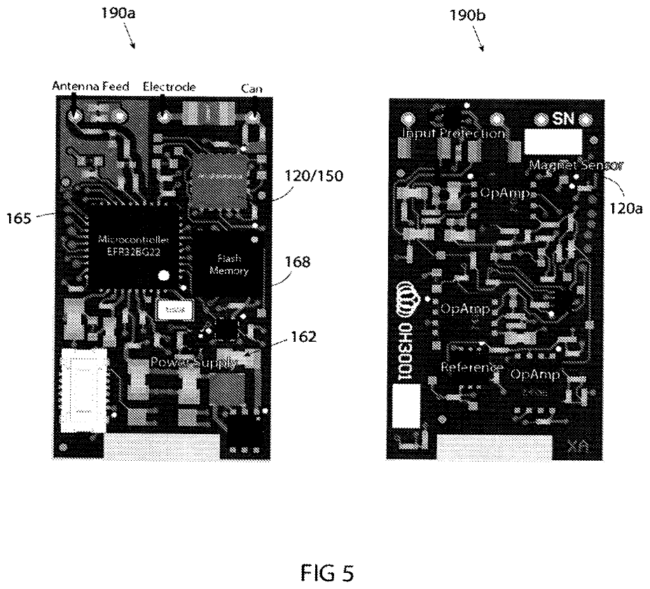

Implantable device 100 can further comprise a module configured to control (e.g. electrically, mechanically, acoustically, and/or fluidly control) one or more functions of device 100, control assembly 160. Device 100 can comprise one or more electronic modules that include control assembly 160 and/or other electronic portions of device 100, such as is described in reference to FIG. 5 herein. Control assembly 160 can comprise a communication module, module 161 shown. Communication module 161 can comprise an antenna and/or other transceiver. Communication module 161 can comprise a wireless communication module, such as a communication module configured to communicate via a wireless protocol selected from the group consisting of: Bluetooth; Bluetooth low energy (BLE); Z-Wave; Near Field Communication (NFC); Wi-Fi; Radio Frequency and combinations of these. In some embodiments, communication module 161 is configured to transmit and/or receive data to and/or from an external device 200 and/or a user device 300. Control assembly 160 further comprises power supply 162. Power supply 162 can comprise one or more batteries, capacitors, and/or other energy storing and/or providing elements. Control assembly 160 can further comprise processor 165. Processor 165 can comprise an electronic module configured to instruct one or more components of implantable device 100 to perform one or more functions. In some embodiments, processor 165 is configured to receive signals from one or more functional elements, as described herein, of implantable device 100. Processor 165 can comprise a data storage medium, memory 168. Memory 168 can be configured to store data recorded by one or more functional elements and received by processor 165.

In some embodiments, processor 165 further comprises one or more algorithms, algorithm 166 shown. Control assembly 160 can comprise one or more electronic elements, electronic assemblies, and/or other electronic components, such as components selected from the group consisting of: memory storage components; analog-to-digital converters; rectification circuitry; state machines; microprocessors; microcontrollers; filters and other signal conditioners; sensor interface circuitry; transducer interface circuitry; and combinations thereof. In some embodiments, control assembly 160 comprises a memory storage component (e.g. memory 168) that includes instructions, such as instructions used by control assembly 160 to perform an algorithm, deliver a control signal, and/or deliver a therapy, each as described herein. Algorithm 166 can be configured to perform one or more computations based on the recorded data received by processor 165 (e.g. from one or more sensors of implantable device 100 or other component of system 10). In some embodiments, communication module 161 is configured to transmit data received by processor 165 to one or more of external devices 200 and/or user devices 300. In some embodiments, algorithm 166 is configured to perform an analysis of the data received by processor 165, and to select a subset of the data to be communicated to device 200 and/or device 300. In some embodiments, algorithm 166 comprises a machine learning algorithm or other artificial intelligence algorithm (“machine learning algorithm” herein), such as is described in reference to FIG. 1 and otherwise herein.

In some embodiments, implantable device 100 further comprises one or more functional elements, functional element 199 shown. Functional element 199 can comprise one, two or more sensors, transducers, and/or other functional elements selected from the group consisting of: a light; a speaker; a user input device such as a button; an optical sensor; a haptic feedback sensor, a drug delivery element; a blood gas sensor; a glucose sensor; a heating element; a cooling element; a vibrational transducer; an electromagnetic field generating transducer; and combinations of these. In some embodiments, functional element 199 of implantable device 100 comprises a wireless charging module. In some embodiments, functional element 199 comprises a tactilely actuated element, such as a switch that can be activate via palpation though the skin of the patient (e.g. a switch configured to activate a function of implantable device 100, such as to turn on the device or initiate a pairing process, such as a Bluetooth pairing process). In some embodiments, functional element 199 can comprise a haptic feedback sensor that is activated with a predetermined touch to the surface of the skin where the device is located (e.g. the user may tap the skin surface region three times in a predetermined manner to activate device 100 to perform a desired recording and/or other function. In some embodiments, functional element 199 comprises one or more elements configured to deliver a therapy, such as an element configured to deliver a drug or other agent to the patient, an element configured to deliver electrical current to the patient (e.g. to pace or defibrillate the heart), and/or an element configured to deliver any form of energy to the patient (e.g. light energy, sound energy; mechanical energy, chemical energy, and/or electromagnetic energy), such as energy configured to deliver a therapy to the patient. In some embodiments, functional element 199 comprises a tactile feedback element.

External device 200 comprises one or more housings, housing 201 shown, surrounding the various components of external device 200. In some embodiments, external device 200 is configured as a “patch-type” device that can be attached (e.g. adhesively attached) to one or more skin surface locations (e.g. proximate the abdomen such as for recording gastrointestinal sounds, proximate the lungs for recording respiration, proximate the fetus of a pregnant patient). One or more components of external device 200 can extend through at least a portion of housing 201, and/or be flush with the surface of housing 201 (as described herein), such as to interact with (e.g. measure a parameter of) tissue and/or fluids (“tissue” herein) proximate the component and/or to interact with (e.g. measure a parameter of) the environment surrounding the patient (e.g. measure the temperature, humidity, pressure, and/or other parameter of air surrounding external device 200 and the patient). In some embodiments, system 10 further comprises a device for securing external device 200 to the patient, wearable garment 202. In some embodiments, wearable garment 202 comprises a strap configured to secure external device 200 to the chest or abdomen of the patient. Alternatively or additionally, wearable garment 202 can comprise a form fitting garment, such as a garment made from elastic synthetic fibers. Wearable garment 202 can include a pocket configured to receive external device 200 and position external device 200 relative to the patient. In some embodiments, wearable garment 202 comprises one or more openings (e.g. an opening in a pocket of wearable garment 202) positioned to allow one or more components of external device 202 to contact the skin of the patient. Additionally or alternatively, external device 200 can be configured to be adhered to the skin of the patient (e.g. when device 200 includes an adhesive surface). In some embodiments, external device 200 comprises a non-rigid (e.g. flexible) device, such as a device comprising at least a flexible portion that is configured to conform to a contour of the patient's body. In some embodiments, external device 200 comprises a patch-like construction, for example a relatively thin and flexible construction (e.g. when the electronics of external device 200 comprise flexible circuit boards and/or low-profile devices), which can be configured to be temporarily adhered to the skin of the patient and flex during patient motion of the attached skin area. External device 200 can comprise a disposable device, such as a device configured to be worn by the patient for a particular period (e.g. 3 days, or one week), and then discarded and replaced. Alternatively or additionally, external device 200 can comprise a replaceable adhesive pad, such that the user can replace the adhesive pad after the first pad's adhesive properties have sufficiently deteriorated.

External device 200 can comprise one or more electrical components, such as first electrode 210a and second electrode 210b shown (collectively or singularly electrode 210 or electrodes 210 herein). Electrodes 210 can comprise at least two electrodes, such as two electrodes positioned on opposite ends of housing 201. At least a portion of each electrode 210 can be positioned outside of housing 201, such that each electrode 210 can be positioned in contact with the skin of the patient. For example, a surface of an electrode 210 can be coplanar with a portion of housing 201, such that the outer surface of external device 200 is relatively smooth (e.g. electrode 210 does not protrude beyond the outer surface of housing 201). Alternatively, the surface of electrode 210 can project from the surface of housing 201, such as to enable enhanced contact between electrode 210 and the skin of the patient when device 200 is positioned against the skin of the patient. In some embodiments, first electrode 210a and/or second electrode 210b each comprise multiple electrodes, such as two, three, or more electrodes. In some embodiments, electrodes 210 are utilized by system 10 to record ECG signals of the patient, as described herein. In some embodiments, external device 200 can provide patient physiologic data that is used in conjunction with patient physiologic data provided by device 100, such that system 10 can provide a more accurate and/or more complete diagnostic result comprising a diagnosis and/or prognosis of one or more medical conditions of the patient. For example, external device 200 can comprise a device configured to provide ECG data to complement ECG recorded by device 100 and/or otherwise improve the diagnostic result provided by system 10. Alternatively or additionally, external device 200 can comprise a device that can be positioned proximate the patient's lungs to provide respiratory data to complement respiratory data recorded by device 100 and/or otherwise improve the diagnostic result provided by system 10 (e.g. by providing higher respiratory auscultation data). Alternatively or additionally, external device 200 can comprise a device configured to be positioned proximate the abdominal region to provide gastrointestinal-related auscultation, and/or proximate a joint for orthopedic-related auscultation, such as to provide data complementary to data provided by implantable device 100 and/or otherwise improve the diagnostic result provided by system 10.

External device 200 can comprise one or more acoustic sensors, sensor 220. Sensor 220 can comprise a microphone, an accelerometer (e.g. an accelerometer configured to record sound signals), and/or other acoustic sensor component. In some embodiments, housing 201 comprises a material with a high acoustic transparency, such that sound can penetrate housing 201 and can be recorded by sensor 220 from within housing 201.

Additionally or alternatively, housing 201 can comprise one or more ports (not shown, but such as membrane covered openings) configured to allow sound to penetrate housing 201. Sensor 220 can comprise multiple acoustic sensors, such as at least two acoustic sensors positioned on opposite ends and/or opposite sides of external device 200.

External device 200 can comprise one or more temperature sensors, temperature sensor 230. At least a portion of temperature sensor 230 can be positioned outside of housing 201, for example, such that at least a portion of temperature sensor 230 is in contact with the patient (e.g. when external device 200 is positioned proximate the skin of the patient). In some embodiments, temperature sensor 230 comprises two or more temperature sensors. For example, temperature sensors 230 can comprise a first temperature sensor configured to record the temperature of the patient, and a second temperature sensor configured to record ambient temperature (e.g. the temperature of the environment surrounding external device 200).

External device 200 can comprise one or more pressure sensitive transducers, pressure sensor 240 shown. In some embodiments, at least a portion of pressure sensor 240 can be positioned outside of housing 201. For example, at least a portion of pressure sensor 240 can extend from housing 201 to contact a portion of the patient's skin (e.g. when device 200 is positioned proximate the skin of the patient). In some embodiments, pressure sensor 240 is configured to record pressure variations proximate the patient (e.g. atmospheric pressure variations surrounding the patient). In some embodiments, pressure sensor 240 comprises two or more pressure sensors.

External device 200 can comprise one or more accelerometers, accelerometer 250 shown. Accelerometer 250 can comprise one or more one-axis, two-axis, and/or three-axis accelerometers. In some embodiments, accelerometer 250 is utilized by system 10 to monitor the activity level of the patient. In some embodiments, accelerometer 250 comprises two or more accelerometers.

External device 200 further comprises a module, control assembly 260, which can be configured to control one or more functions of device 200. Control assembly 260 can comprise communication module 261. Communication module 261 can comprise an antenna and/or other transceiver, such as for transmitting data to and/or from external device 200. Communication module 261 can comprise a wireless communication module, such as a communication module configured to communicate via a wireless protocol selected from the group consisting of: Bluetooth; Bluetooth low energy (BLE); Z-Wave; Near Field Communication (NFC); Wi-Fi, and combinations of two or more of these. in some embodiments, communication module 261 is configured to transmit and/or receive data to and/or from implantable device 100 and/or user device 300. Control assembly 260 can further comprise power supply 262. Power supply 262 can comprise one or more batteries, capacitors, and/or other energy storing and/or providing elements. Control assembly 260 can further comprise processor 265. Processor 265 can comprise a microcontroller or other processer that can be configured to instruct one or more components of external device 200 to perform one or more functions. In some embodiments, processor 265 is configured to receive signals from one or more functional elements of external device 200. Processor 265 can comprise a data storage medium, memory 268. Memory 268 can be configured to store data recorded by one or more functional elements and received by processor 265.

In some embodiments, processor 265 further comprises one or more algorithms, algorithm 266 shown. Control assembly 260 can comprise one or more electronic elements, electronic assemblies, and/or other electronic components, such as components selected from the group consisting of: memory storage components; analog-to-digital converters; rectification circuitry; state machines; microprocessors; microcontrollers; filters and other signal conditioners; sensor interface circuitry; transducer interface circuitry; and combinations thereof. In some embodiments, control assembly 260 comprises a memory storage component (e.g. memory 268) that includes instructions, such as instructions used by control assembly 260 to perform an algorithm, deliver a control signal, and/or deliver a therapy, each as described herein. Algorithm 266 can comprise one or more algorithms that can be configured to perform one or more calculations based on the recorded data received by processor 265. In some embodiments, communication module 261 is configured to transmit data received by processor 265 to one or more of implantable devices 100 and/or user devices 300. In some embodiments, algorithm 266 is configured to perform one or more analyses of the data received by processor 265, and to select a subset of the data to be communicated to device 100 and/or device 300. In some embodiments, algorithm 266 comprises a machine learning algorithm or other artificial intelligence algorithm.

In some embodiments, external device 200 further comprises one or more sensors, transducers, and/or other functional elements, functional element 299 shown. Functional element 299 can comprise one, two, or more elements selected from the group consisting of: a light; a speaker; a user input device such as a button; an optical sensor (e.g. an optical sensor for measuring oxygen saturation and/or Vmax); a drug delivery element; a heating element; a cooling element; a vibrational transducer; an electromagnetic field generating transducer; a blood gas sensor; a glucose sensor; and combinations of these. Functional element 299 can comprise a wireless charging module. In these embodiments, external device 200 can be configured to provide a source of energy to implantable device 100, such as to charge power supply 162 of implantable device 100 (e.g. when power supply 162 comprises a rechargeable power supply). Power supply 262 can comprise a rechargeable power supply. Power supply 262 can be configured to be recharged wirelessly and/or via a charging port such as a USB charging port.

As described herein, external device 200 can be configured to transmit power and/or data to, and/or receive data from, one or more implantable devices 100. In some embodiments, external device 200 comprises one, two, or more devices that are configured to: retrieve data from the at least one implantable device 100; transmit software update data to an implantable device 100; and/or delivery electrical energy to (e.g. to recharge and/or power) at least one implantable device 100.

User device 300 comprises one or more housings, housing 301 shown, surrounding the various components of user device 300. User device 300 can comprise user interface 310. User interface 310 can comprise one or more of: a display, such as a touch screen display; one or more user input devices, such as one or more buttons; a speaker; a microphone; a tactile feedback element; and combinations of these. User device 300 can comprise power supply 362. Power supply 362 can comprise one or more batteries, capacitors, and/or other energy storing and/or providing elements. In some embodiments, user device 300 comprises one or more of the following devices: a smart phone; a smart watch; a tablet; a desktop computer; a laptop computer; and combinations of these. In some embodiments user device 300 comprises two or more devices configured to communicate with implantable device 100 and/or external device 200, as well as server 400. For example, a user of system 10 may use both a smartphone and a laptop computer to communicate with devices 100 and/or 200, as well as server 400. In some embodiments, system 10 comprises a software application, app 3100. App 3100 can be provided via server 400 for download and installation to a user device, such as a smart phone. App 3100 can be configured to enable the user device to perform one or more functions of user device 300 described herein. For example, app 3100 can include software including one or more algorithms (e.g. algorithm 366 described herein) that are configured to be executed via a processor of the user device (e.g. processor 365 described herein).

In some embodiments, user device 300 comprises one or more functional elements, functional element 399 shown. Functional element 399 can comprise one, two, or more elements selected from the group consisting of: a light; a speaker; a user input device such as a button; an accelerometer; a temperature sensor; a pressure sensor; a GPS sensor; a proximity sensor, such as a proximity sensor using NFC or Bluetooth; an optical sensor, such as an IR sensor and/or a camera; a fingerprint sensor; an identification element (e.g. an RFID); and combinations of these.

User device 300 further can comprise a module, control assembly 360, which can be configured to control one or more functions of device 300. Control assembly 360 can comprise a communication module, module 361 shown. Communication module 361 can comprise an antenna and/or other transceiver. Communication module 361 one can comprise a wireless communication module such as a communication module configured to communicate via a wireless protocol selected from the group consisting of: Bluetooth; Bluetooth low energy (BLE); Z-Wave; Near Field Communication (NFC); Wi-Fi; Radio Frequency; and combinations of these. In some embodiments, communication module 361 is configured to transmit and/or receive data to and/or from an implantable device 100 and/or an external device 200. Additionally, communication module 361 can be configured to transmit and/or receive data to and/or from server 400, such as via a network, for example, the Internet. Control assembly 360 can further comprise processor 365. Processor 365 can comprise an electronic module configured to instruct one or more components of user device 300 to perform one or more functions. In some embodiments processor 365 is configured to receive data from implantable device 100 and/or external device 200. Processor 365 can be further configured to receive data from functional element 399 and/or other components of user device 300. Processor 365 can comprise a data storage medium, memory 368. Memory 368 can be configured to store data received from implantable device 100, external device 200, and/or other components of user device 300. Memory 368 can store instructions, such as instructions used by processor 365 to perform an algorithm (e.g. algorithm 366 described herein), deliver a control signal, and/or deliver a therapy, each as described herein.

In some embodiments, processor 365 further comprises one or more algorithms, algorithm 366 shown (e.g. a machine learning algorithm). Algorithm 366 can comprise one or more algorithms that are configured to perform one or more computations based on the data received by processor 365. In some embodiments, communication module 360 is configured to transmit data received by processor 365 to server 400. Algorithm 366 can be configured to perform an analysis of the data received by processor 365. In some embodiments, a subset of the data received by processor 365 is transmitted to server 400, and/or an analysis of the data performed by algorithm 366 is transmitted to server 400.

Server 400 can comprise a communication module, module 461 shown, which can be configured to communicate with a user device 300 and/or other devices of system 10 via a communication network, such as the Internet. Devices 100, 200, and/or 300 can comprise devices local to the patient (e.g. implanted in, worn on, carried with, and/or installed at a patient's home or office). Server 400 can comprise a device remote from the patient (or remote from a set of patients), such as when server 400 is a cloud-based server and/or a server hosted by the provider of system 10. Server 400 can comprise a processor 465 and one or more databases of information, data storage 469. System 10 can include a single server 400 (e.g. one or more physical server devices comprising a single computational and/or data storage unit), and include multiple sets of one or more of devices 100, 200, and/or 300, each set provided to a patient among a group of patients (P_{GROUP}) utilizing system 10.

Processor 465 can include one or more algorithms, algorithm 466 shown. Processor 465 can comprise a memory storage component configured to store instructions used to perform algorithm 466. Algorithm 466 can comprise one or more algorithms that are configured to perform one or more computations based on data transmitted to server 400 from one or more patients of P_{GROUP}. One or more of algorithms 166, 266, 366, and/or 466 can include a biasing function, bias 167, 267, 367, and/or 467, respectively. Each of bias 167, 267, 367, and/or 467 can cause the associated algorithm to: produce an output that tends towards a particular type of outcome (e.g. such as a bias towards false positives and/or false negatives); apply a safety margin (e.g. a safety margin configured to reduce the likelihood of an adverse event); apply a filter (e.g. a filter configured to remove outlier or other inapplicable data); and/or apply another biasing function.

Processor 465 can further include a neural network, machine learning architecture, and/or other artificial intelligence architecture, AI 468. AI 468 can be utilized by processor 465 singularly and/or in conjunction with algorithm 466 to process data transmitted to server 400.

As described herein, system 10 can be configured to record data representative of one or more patient parameters via one or more sensors or other functional elements of devices 100, 200, and/or 300, and via one or more processors of system 10 to analyze the recorded data to provide a diagnostic result comprising one or more diagnoses and/or prognoses of one or more medical conditions (e.g. one or more diseases and/or disorders) of the patient (e.g. a patient of P_{GROUP}). As used herein, a processor of system 10 can include one or more of processors 165, 265, 365, and/or 465. A patient device of system 10 can include one or more of devices 100, 200, and/or 300. Data collected from a single patient can be compared with data of any other patient of P_{GROUP} via processor 465 of server 400 (e.g. when patient data from multiple patients of P_{GROUP} is transmitted to server 400 and stored in data storage 469 for analysis and/or comparison).

Clinician device 500 can comprise a computing device configured to allow a clinician of a patient of system 10 to review data collected and/or produced by system 10. Clinician device 500 can be further configured to enable the clinician to communicate directly with user device 300, such as to modify a setting of user device 300 and/or devices 100 or 200. Additionally or alternatively, clinician device 500 can communicate directly with a device 100 and/or 200. Clinician device 500 can be configured to communicate remotely with server 400 and/or user device 300 via a communication network such as the Internet. In some embodiments, clinician device 500 can communicate locally (e.g. when the patient is in the clinician's office) with any of devices 100, 200, and/or 300, such as via Bluetooth. Clinician device 500 can comprise a device similar to user device 300. Clinician device 500 can comprise a desktop computer, a laptop computer, a smart phone, a tablet, and/or other computing device.

In some embodiments, system 10 is configured to monitor the ECG of a patient. For example, implantable device 100 can record ECG signals via first electrode 110a and second electrode 110b. In some embodiments, ECG data is recorded from both implantable device 100 and external device 200. Alternatively or additionally, system 10 can be configured to record a “single lead ECG”, such as an ECG recorded from any of electrodes 110a, 110b, 210a, and/or 210b. ECG data can be analyzed by a processor of system 10 for one or more of the following objectives: to produce a heartrate histogram; to determine heartrate trends over a period of time; to observe and/or count one or more acute events, such as an intermittent arrythmia; to detect an embolic event; to improve P-wave discrimination in atrial fibrillation monitoring; to produce an AFIB burden report; to improve QRS discrimination; to determine QT interval for ventricular arrhythmia monitoring; to monitor ST elevation; to determine heart rate variability overtime; and combinations of these. In some embodiments, system 10 is configured to measure Impedance Cardiography (ICG) via one or more of electrodes 110a, 110b, 210a, and/or 210b. In some embodiments, signals recorded from first electrode 110a and second electrode 110b are analyzed by system 10 to measure electrical resistance (e.g. tissue resistance) between the two electrodes 110. System 10 can be implemented to conduct a 120-hour continuous Holter monitor-like test (e.g. to measure the heart activity of the patient, such as the rate and rhythm of the heart). In some embodiments, system 10 is configured to analyze a data set of both ECG data and PCG data, such as to produce electromechanical activate time (EMAT) data, such as to produce a diagnostic result comprising a diagnosis of heart failure of the patient.

In some embodiments, system 10 is configured to monitor one or more patient body sounds. For example, system 10 can record a PCG (e.g. a recording of cardiac sounds) from sensors 120 and/or 220. Recorded cardiac sound data can be analyzed by a processor of system 10 to identify S1, S2, S3, and S4 sounds. In some embodiments, system 10 is configured to record a PCG over a first duration, and to repeat the recording for the first duration periodically over a second duration. For example, system 10 can be configured to record a 20 second PCG every one to two hours (e.g. for several days up to 5 years). A processor of system 10 can be configured to compare sets of these periodic recordings, such as to analyze trends and/or to diagnose the patient.

System 10 can record lung sounds from sensors 120 and/or 220. In some embodiments, the location where external device 200 is positioned relative to the patient (e.g. where device 200 is adhered to the patient) is determined based on an optimal location for monitoring lung sounds. In some embodiments, the position is determined such that external device is positioned opposite a body organ (e.g. the lung) from implantable device 100, such that signals can be recorded on either side of and/or through the organ.

System 10 can be configured to record vocal sounds from one or more sensors 120 and/or 220. A processor of system 10 can be configured to analyze vocal sounds to enable voice control of one or more components of system 10. In some embodiments, sensor 120 of implantable device 100 is configured to record vocal sounds spoken by the patient (e.g. as heard from within the body cavity, at one or more locations under the patient's skin). Additionally or alternatively, sensor 120 can be configured to record vocal sounds from a person or persons proximate the patient (e.g. as heard through the body cavity). System 10 can be configured to recognize these vocal sounds given distortion caused by the body cavity (e.g. via a compensation routine which accounts for tissue types through which the sounds traverse). For example, algorithm 166 can comprise a speech recognition algorithm configured to recognize these distorted vocal sounds, and/or algorithm 166 can comprise a standard voice recognition algorithm (e.g. used with Amazon device such as Alexa) and bias 167 is configured to account and compensate for tissue and other body cavity distortions of the recorded vocal sounds. In some embodiments, algorithm 166, and/or another algorithm of system 10, is configured to identify the person creating the vocal sounds (e.g. differentiate the patient versus the patient's family member or clinician).

In some embodiments, system 10 is configured to monitor the body temperature of the patient. For example, system 10 can record the patient's internal body temperature proximate implantable device 100 using temperature sensor 130. Additionally or alternatively, system 10 can be configured to monitor temperature surrounding the patient (e.g. ambient temperature proximate the patient), for example using temperature sensor 230. In some embodiments, system 10 is configured to monitor the temperature of the patient continuously for a period of time, for example continuously for at least 24 hours, 48 hours, 72 hours, 96 hours, and/or 90 days. In some embodiments, system 10 is configured to identify an increase in body temperature as it is related to the patient's heart failure condition and/or ovulation.

In some embodiments, system 10 is configured to record a pressure measurement, such as a pressure measurement of the patient (e.g. blood pressure and/or other fluid pressure) and/or of the patient's environment (e.g. pressure of the atmosphere surrounding the patient, such as to compensate for patient's that live at high altitudes). In some embodiments, system 10 is configured to record a seismocardiogram (SCG) using pressure sensor 140.

In some embodiments, system 10 is configured to monitor the posture of the patient by analyzing data recorded by accelerometers 150 and/or 250, and/or another one or more sensors of system 10. For example, system 10 can monitor the posture of the patient while the patient is sleeping, such as to identify the sleep position of the patient (e.g. sleeping on the left side or right side). System 10 can analyze the identified sleep position along with other data recorded by system 10, for example to identify cardiac changes based on the detected sleep position.

In some embodiments, system 10 is configured to compare a first set of data from a first time period to a second set of data from a second time period. For example, system 10 can analyze heart sound data and accelerometer data recorded during the first and second time periods, identify the patient position during the time periods, and use the patient position data in the analysis to produce a diagnostic result (e.g. since heart sound data changes with body position). For example, heart sound data can be interpreted based on the patient position during which the heart sound data was recorded, such as when the diagnostic result is based on patient position.

In some embodiments, system 10 is configured to monitor chest movement (e.g. up and down movement caused by respiration, as determined by an accelerometer or other functional element of system 10, as described herein), such as to produce a diagnostic result based on chest movement of the patient.

System 10 can be configured to monitor the activity level of the patient, for example to identify periods of exercise, moderate activity, and/or sedentary periods (e.g. as determined by an accelerometer or other functional element of system 10, as described herein), such as when a diagnostic result is determined based on the activity level of the patient.

In some embodiments, functional elements 199, 299, and/or 399 comprise one, two, or more sensors, transducers, and/or other functional elements selected from the group consisting of: optical sensor; blood gas sensor; blood glucose sensor; an impedance sensor; a perspiration sensor, such an impedance sensor configured to vary based on the moisture level of the skin of the patient; an alcohol sensor, such as a blood alcohol sensor; a biochemical sensor; a haptic transducer, such as a vibrational transducer; an acoustical transducer, such as a microphone or a speaker; an electrical sensor and/or transducer, such as an electrode configured to record and/or transmit electrical signals; an electrochemical sensor; an ion selective sensor, such as a sensor including an ion selective electrode; a temperature sensor; blood gas sensor; a strain gauge; a pressure sensor; a GPS sensor; a heating element; a cooling element such as a thermoelectric cooling element; and combinations of these. In some embodiments, a diagnostic result produced by system 10 is based on data collected by one or more of these functional elements.

In some embodiments, system 10 is configured to analyze optical data (e.g. optical data recorded by a functional element 199, 299, and/or 399), such as to diagnose oxygen saturation and/or heart rate of the patient, such as when a diagnostic result produced by system 10 is based upon oxygen saturation and/or heart rate.

In some embodiments, system 10 comprises two or more sensors configured to measure a single parameter (e.g. a single physiologic parameter of the patient and/or a single patient environment parameter), such as two, three or more sensor-based functional elements configured to produce data that is compared to determine that the data produced by a sensor is not accurate (e.g. the sensor is broken or otherwise producing unacceptable data). In some embodiments, three or more sensors are used, and data that correlates between two or more sensors is used (e.g. used by an algorithm of system 10 to produce a diagnostic result), while data from a single sensor that does not correlate with the other data is thrown out (e.g. not used by an algorithm of system 10 to produce a diagnostic result).

One or more device of system 10 can communicate with one or more other devices of system 10 via one or more communication protocols, as described herein. In some embodiments, devices 100 and/or 200 are configured to transmit sets of recorded data to user device 300 at a fixed or variable interval, such as once every set number of hours (e.g. once every hour to twelve hours). For example, implantable device 100 can be configured to record data for a period of time (e.g. one hour), and to transmit the data recorded in that hour to user device 300 in a single communication (e.g. a communication of a relatively short time period as compared to the time period in which the data was collected). Alternatively, devices 100 and/or 200 can be configured to continuously transmit recorded data to user device 300. In some embodiments, devices 100 and/or 200 are configured to transmit a first category of data (e.g. data determined by an algorithm of system 10 to be urgent) whenever a connection to user device 300 is available, and to only transmit a second data type on a schedule (e.g. data determined to be non-urgent is transmitted on a pre-determined schedule). In some embodiments, devices 100 and/or 200 are configured to record and store data (e.g. as memory permits) until the patient requests, via user device 300, to download the recorded data from devices 100 and/or 200 to user device 300. In some embodiments, data is transmitted when memory storage of a device 100 or other device, as described herein, is at a threshold level (e.g. a maximum memory storage capacity is being reached)

In some embodiments, user device 300 is configured to transmit recorded data to server 400, such as via a communication network such as the Internet. Server 400 can be configured to analyze the recorded data and transmit the results of the analysis back to the patient. In some embodiments, server 400 is configured to perform cloud-based analysis of the patient data, such as an analysis that utilizes a machine learning algorithm of system 10.

In some embodiments, system 10 analyzes data recorded by multiple types of sensors described herein (e.g. system 10 analyzes data from multiple sensors to produce a diagnostic result for the patient). For example, system 10 can analyze data recorded from two, three, four, or more of sensor types selected from the group consisting of: electrodes; acoustic sensors; temperature sensors; pressure sensors; accelerometers; blood gas sensors; glucose sensors; perspiration sensors; activity level sensors; GPS sensors; light sensors; and combinations of these. Analyzing data from multiple sensor types can enable system 10 to more accurately diagnose a patient than by analyzing a single data type (e.g. to generate a more accurate diagnosis and/or to generate a different type of diagnosis than otherwise possible with a single type of data). System 10 can analyze the recorded data to determine information selected from the group consisting of: amplitude and/or frequencies of P waves, QRS waves, premature ventricular contractions (PVC), QT relations, ST relations; amplitude and/or frequencies of S1, S2, S3, and/or S4 signals; Electro Mechanical Activation Time (EMAT); Systolic Disfunction Index (SDI); the number of apnea events to occur in a period of time, activity level; body posture; body position (e.g. to compensate corresponding heart sounds); and combinations of these.

In some embodiments, implantable device 100 and external device 200 each record the same type of data to be analyzed by system 10 to produce complementary data sets, in other words two independent sets of similar data, for example an internal ECG and an external ECG to be analyzed collectively by system 10. In these embodiments, the diagnostic result produced by system 10 can be based upon a comparison of the two data sets.

System 10 can be configured to determine one or more patient parameters selected from the group consisting of: diastolic disfunction index; peak endocardial acceleration; comprehensive cardiac data; and combinations of these. System 10 can perform an analysis of one or more of these parameters to provide a diagnostic result that provides a diagnosis and/or prognosis of one or more medical conditions of the patient.

System 10 can produce a diagnostic result that diagnoses and/or prognoses (singly or collectively “diagnose” herein) the patient's breathing by monitoring lung sounds via one or more of sensor 120 and/or 220 (e.g. a sensor 120 comprising accelerometer 150 and/or a sensor 220 comprising accelerometer 250). Alternatively or additionally, system 10 can be configured to produce a diagnostic result based on monitored chest movement via accelerometer 150 and/or 250. By analyzing lung sounds and chest movement in conjunction, system 10 can provide a more accurate diagnosis and/or prognosis (singly or collectively “diagnosis” or “diagnostic result” herein) than analyzing either set of data alone.

In some embodiments, system 10 can produce a diagnostic result for the patient by analyzing electrical signals recorded from both electrodes 110 (internal) and 210 (external). For example, system 10 can generate a wide vector ECG, such as an EKG reading correlated to a two to six lead ECG, for example when at least one of the ECG leads comprises an electrode 110 of implantable device 100, and at least one of the ECG leads comprises an electrode 210 of external device 200. In some embodiments, system 10 is configured to diagnose fluid in the lungs of the patient by analyzing transthoracic impedance measured between an electrode 110 and an electrode 210.

In some embodiments, system 10 provides (e.g. via server 400) one or more patient data reports (e.g. including one or more diagnostic results produced by system 10). These reports can be available to a clinician of a patient and/or a patient of system 10, such as via clinician device 500 and/or user device 300. For example, server 400 can provide an Internet-based web portal, where a clinician can log in and access data and/or reports relating to the particular patients of that clinician using system 10 (e.g. such as when a clinician will log in two to four times per week). In some embodiments, the reports include diagnostic results that indicate data trends, as identified by an algorithm of system 10. In some embodiments, the trends are based on a single patient's data, and/or the trends are based on data collected from multiple patients. In some embodiments, system 10 produces one or more diagnostic data reports selected from the group consisting of: an arrhythmia and activity report; a heart sound and cardiac report; a diagnostic report; a prognostic report; a trending report; and combinations of these.

In some embodiments, an algorithm of system 10 is configured to perform a trend analysis of the collected data, such as to produce diagnostic results that identify changes (e.g. minute changes) in the data over time, and/or to identify other trends in the data. For example, a trend analysis can determine subtle changes in S1, S2, S3, and/or S4 sounds recorded over time. An algorithm of system 10 can perform an analysis of the amplitude, frequency, and/or timing of any or all of S1, S2, S3 and S4 data. The amplitude and frequency of these heart sounds can be analyzed, and the number of instances of these sounds (e.g. S1, S2, S3 and/or S4 sounds) can be counted (e.g. and compared to previous events in a comparable time frame). This data can be comparatively analyzed by system 10 to similar data recorded daily, weekly, and/or monthly, for example over a period of up to 3-5 years. As another example, a diagnostic result comprising a trend analysis can determine a template for P-wave discrimination in ECG data by analyzing ECG data over time. This template can be used to filter P-wave signals from ECG data such that system 10 can better analyze the ECG data for atrial activity, such as to diagnose an atrial arrythmia of the patient.

The one or more algorithms (e.g. algorithms 166, 266, 366, and/or 466) of system 10 described herein, for brevity, are also referred to herein, singly or collectively, as algorithm 1066. Algorithm 1066 can be performed by a control unit and/or processor of system 10 via instructions stored in a memory storage component of system 10. Algorithm 1066 can be configured to analyze data collected by the various sensors of system 10 described herein to monitor patient health, diagnose a patient condition (i.e. produce a diagnostic result), and/or predict an adverse event (e.g. a stroke and/or a heart attack). In some embodiments, system 10 is configured to monitor and/or analyze data comprising ECG data; heart sound data; and at least one additional physiologic parameter of the patient. ECG data (e.g. ECG data recorded by system 10) has multiple segments that represent atrial and ventricular activities, represented by P, Q, R, S, T, U, as shown in FIG. 3A. The time intervals between each segment represent cardiac cycle and regularity. For example, an elongated PR interval may represent possible disease of an electrical pathway. Irregular RR interval may represent one or more cardiac arrhythmias. Elevated ST segment may indicate a heart attack. Heart sounds (e.g. heart sounds recorded by system 10) also provide diagnostic information. Various recorded sounds can represent the presence of various mechanical anomalies within the heart, and these heart sounds can be analyzed by system 10 in order to provide a diagnosis and/or prognosis (“diagnosis” herein) of the patient. Heart sounds recorded by system 10 can be location specific, as described below and otherwise herein. Mitral, tricuspid, aortic, pulmonic, and xiphoid regions each have a slightly varied sound signature. Applicant has performed studies in which digital stethoscope recordings from three human patients were recorded. Data collected included digital recordings representing heart sounds in the mitral area, tricuspid area, aortic area, pulmonic area, and xiphoid area, as shown in FIGS. 3D-3F. These and other heart sound variations (e.g. as recorded by an accelerometer and/or other sound recording components of system 10) can be detected by an algorithm 1066 of system 10. Algorithm 1066 can utilize the known location of the heart sound to analyze subtle changes in heart sounds. In some embodiments, data collected from an accelerometer (e.g. accelerometer 150 comprising a three-axis accelerometer and/or other accelerometer) can be analyzed by algorithm 1066, such as an analysis performed also using heart sound data, blood pressure data, blood flow data, chest motion data, patient position data, patient posture data, patient activity data, EKG data, temperature data, and/or other data recorded by system 10. Heart muscle has a similar constitution as firm gelatinous muscle, and the heart moves as the body of the patient transitions between different orientations and postures. These body position changes result in slightly varying heart sounds that can be differentiated by algorithm 1066. For example, a patient position and/or state (e.g. sleeping, sitting, walking, or lying down on their left side or right side), can be determined by system 10 (e.g. via accelerometer 150 or other sensor of system 10), and such data included in an analysis performed by an algorithm 1066.

System 10 can record patient data over a period of time, for example days, weeks, months, and/or years, and algorithm 1066 can be configured to perform an analysis using previously recorded patient data as well as current patient data, for example, data recorded within an hour, such as within five minutes, or near real time (e.g. within seconds) of the analysis by algorithm 1066. System 10 can record patient data per pre-programmed parameters, and/or based on manual demand by a user, as described herein. Implantable device 100 provides an advantage over external recording devices, as the location of device 100 doesn't change as data is recorded over time, thus providing consistent and reliable data over long periods of time. Heart sound data, ECG data, and/or other data recorded by implantable device 100 remain consistent for the duration of the implant, resulting in reliable, long-term data that can be compared with other recorded and/or otherwise known patient parameters. Algorithm 1066 can be configured to assess the morphology of each ECG segment, and can be configured to detect a subtle change that may be recorded over days, weeks, months, and/or years. Algorithm 1066 can be configured to assess the P, Q, R, S, T, U wave of each ECG segment, and its related morphology, amplitude, intensity, timing cycle, and/or frequencies. Additionally or alternatively, system 10 can monitor varying ECG data of diastole and/or systole and its relation to valvular closing sounds. In some embodiments, algorithm 1066 can include breathing (e.g. including subtle changes to labored breathing), voice (e.g. voice changes), and/or body posture and position data in one or more analyses. As the cardiac function of the patient changes, algorithm 1066 can assess the morphology (e.g. angle, slope, slew), amplitude, and frequencies of each recorded ECG signal.

Algorithm 1066 can assess S1, S2, S3, and S4 heart sound signatures, and the related morphology, amplitude, intensity, timing cycle, and/or frequencies of each signature. Algorithm 1066 can assess the varying sounds of diastole and/or systole and their relationship to valvular closing sounds, as well as the impact of patient breathing and/or body posture and position. For example, the S2 signature can include data representing the closure of the aortic and/or pulmonic valves. Within the S2 signature, algorithm 1066 can be configured to discern the A2 signature (closure of the aortic valve), and the P2 signature (closure of the pulmonic valve). This split can be complicated by expiration and inspiration sounds of patient respiration. Further differentiation can be present, and detected by analysis of signals produced by one or more sensors 120 of system 10, as cardiac function declines. In some embodiments, the S3 signature can be an important indicator of systolic heart failure, as the myocardium is usually weaker (or overly compliant), resulting in a dilated left ventricle. S1 is normally a single sound because mitral and tricuspid valves closure can occur almost simultaneously. However, system 10 can be configured to elongate (e.g. magnify the timeline of) the S1 signature, such as to separate mitral and tricuspid sound signatures, such as to assess these sound signatures and correspondingly assess the performance of each valve. S2 represents closure of the aortic (A2) and pulmonic (P2) valves. In a diseased state like heart failure, system 10 can differentiate a split S2 as the timing of valve closure is misaligned. Furthermore, system 10 can differentiate S2 sounds that occur in expiration versus inspiration (e.g. associating a widening S2 during inspiration). System 10 can be configured to identify a widening S2 as being associated with pulmonary stenosis. Alternatively or additionally, system 10 can associate atrial septal defects with a widening of S2 since valvular diseases and other adverse heart conditions can be associated with the mistiming of aortic valve (A2) and pulmonic valve (P2). System 10 can associate abnormal heart sounds in S3 with systolic heart failure. S3 is produced during passive left ventricular filling when blood strikes a compliant Left Ventricle, and a particular S3 recording (e.g. an abnormal recording) can be associated by system 10 with a weakened left ventricle. S4 heart sound occurs during active left ventricle filling when atrial contraction forces blood into the left ventricle. System 10 can be configured to determine if S4 heart sounds are associated with a noncompliant left ventricle. This noncompliance is an important sign of diastolic heart failure and/or active ischemia.

Algorithm 1066 can be configured to analyze at least both ECG and heart sounds to assess the electrical and mechanical function of the heart, as an analysis of ECG alone may not effectively assess the true, current cardiac condition of the patient. Algorithm 1066 can be further configured to analyze the timing between each signal to assess comprehensive cardiac function. As shown in FIG. 3B, an ECG can be recorded indicative of relatively normal cardiac function, and a recorded PCG (e.g. a simultaneously recorded PCG) can be indicative of pathologies (Split S2, presence of S4, and/or presence of S3). Based on the relationships between ECG and PCG, algorithm 1066 can analyze complex timing sequences, such as Electromechanical Activation Time (EMAT), Left Ventricular Systolic Time (LVST), Systolic Timing Interval (STI), Pre Ejection Period (PEP), Left Ventricular Ejection Time (LVET), Isovolumetric Contraction/Relaxation Time (IVCT, IVRT) and many other systolic and diastolic time intervals, as shown in FIG. 3C.

Algorithm 1066 can be configured to analyze multiple aspects of the ECG and PCG morphology, as well as timing, such as an analysis that is based on body posture and/or orientation (e.g. as the heart sounds have marked variations associated with different body postures and orientations). Accordingly, algorithm 1066 can analyze ECG morphology and timing cycle variations taking into account data collected from an accelerometer (e.g. accelerometer 150 comprising a three-axis accelerometer). Additionally or alternatively, algorithm 1066 can analyze PCG sound signature morphology and timing cycle variations taking into account data collected from an accelerometer (e.g. accelerometer 150 comprising a three-axis accelerometer). In some embodiments, algorithm 1066 is configured to analyze both ECG and PCG data (e.g. timing) and associated variations, such as by taking into account data collected from an accelerometer (e.g. accelerometer 150 comprising a three-axis accelerometer used to determine body position and/or changes in body position).

In some embodiments, implantable device 100 comprises one or more relatively small implants configured to interact with (e.g. communicate with) external device 200 (e.g. one or more external devices 200), for example when external device 200 comprises a wearable device. One or more implantable devices 100 can each comprise a length of no more than 7.5 mm, such as a length of about 5.5 mm. In some embodiments, an implantable device 100 is configured to be implanted in the inner wrist, for example when external device 200 comprises a watch and/or portion of a watch band (e.g. to be positioned proximate an implantable device 100 that has been implanted in the inner wrist). Alternatively or additionally, one or more implantable device 100 can be configured to be implanted proximate the patient's ear, such as to be powered by an external device 200 comprising eyeglasses (e.g. smart glasses), headphones, earbuds, and/or other head-worn device.

Alternatively or additionally, one or more implantable device 100 can be configured to be implanted proximate an artery of the patient (e.g. a carotid artery) and/or a vein of the patient, such as to be powered by an external device 200 comprising a patch positioned on the patient's skin proximate that implantable device 100. Implantable device 100 (e.g. each implantable device 100) can be configured to receive power from external device 200, such as power received via a wireless link. In some embodiments, implantable device 100 is configured to be implanted behind the ear and receive power from an external device 200 comprising glasses (e.g. glasses including a transmitter or receiver included in either or both temple tips of the glasses). Implantable device 100 can be configured to monitor a patient parameter, such as temperature, oxygen saturation, body position (e.g. via a three-axis accelerometer), impedance, pulse, and/or pulse pressure. Implantable device 100 can be configured to be implanted in the abdomen, proximate an arterial pulsing area, in the lower leg (e.g. to monitor diabetic patients), proximate a joint, and/or in the scalp of the patient.

Algorithm 1066 can be configured to monitor for changes over time in recorded patient data to detect and/or otherwise diagnose a patient disease or adverse event. For example, algorithm 1066 can be configured to detect an interval change above a threshold, for example a change above a threshold within a time period. For example, a QRS-to-QRS interval change of at least 30% within 2 seconds may be determined by an algorithm 1066 to indicate a state of atrial fibrillation. Algorithm 1066 can be configured to detect an amplitude change above a threshold, for example a change above a threshold over a time period. For example, a decrease in QRS amplitude of at least 40% for at least 2 seconds can be determined by an algorithm 1066 to indicate a state of ventricular fibrillation. In some embodiments, system 10 is configured to allow a clinician to enter and/or modify this threshold. Algorithm 1066 can be configured to monitor the amplitude and/or frequency of heart sounds over a time period, for example a 24-hour time period. For example, a change of at least 5% in amplitude or frequency of heart sounds in a 24-hour period can be determined by an algorithm 1066 to indicate an adverse patient condition.

System 10 can comprise functional element 99 as shown. In some embodiments, functional element 99 comprises a magnet or other magnetic field generating component (“magnet” herein). In these embodiments, device 100 can comprise a magnetic sensor configured to detect the presence of a magnet-based functional element 99 that is positioned (e.g. by the patient) proximate device 100, as described herein in reference to FIG. 5. Functional element 99 can comprise one, two, or more elements selected from the group consisting of: a light; a speaker; a user input device such as a switch; an optical sensor (e.g. an optical sensor for measuring oxygen saturation and/or Vmax); a drug delivery element; a heating element; a cooling element; a vibrational transducer; an electromagnetic field generating transducer; a blood gas sensor; a glucose sensor; and combinations of these.

Referring now to FIG. 4, a system comprising multiple implantable sensing devices is illustrated, consistent with the present inventive concepts. System 10 can comprise one, two, three, or more devices 100 for recording patient physiologic information, as described herein. For example, system 10 can comprise, one, two, three, or more of device 100a, 100b, 100c, 100d, 100e, 100f, 100g, 100h, 100i, 100j, and/or 100k, each implanted in a particular anatomical location of a patient as shown. Each device 100 can be configured to record sound, electrical activity, force, pressure (e.g. blood pressure), flow (e.g. blood flow), temperature, displacement (e.g. tissue movement), and/or other physiologic information of the patient as described herein.

System 10 can comprise one or more devices 100 that are implanted proximate an artery, such as to record at least arterial pressure and/or to record other physiologic information related to an artery and/or blood flow within an artery. In some embodiments, system 10 comprises a device 100 that is implanted proximate a posterior auricular artery (e.g. device 100a shown implanted behind an ear of the patient). System 10 can comprise a device 100 that is implanted proximate a carotid artery (e.g. device 100b shown implanted in the patient's neck). System 10 can comprise a device 100 that is implanted proximate a radial artery (e.g. device 100j and/or 100l implanted proximate a wrist of the patient). System 10 can comprise at least a device 100 that is implanted proximate a brachial artery, such as a device 100 implanted proximate a shoulder (e.g. device 100c shown) and/or within the upper arm of the patient.

System 10 can comprise one or more devices 100 that are implanted to record sounds emanating from a lung of the patient (e.g. device 100d, 100e, and/or 100c).

System 10 can comprise a device 100 implanted in the upper chest (e.g. device 100c shown implanted in the left upper chest), and a device implanted in the lower abdomen (e.g. device 100h shown implanted in the left lower abdomen), such as to record at least EEG data with a wide vector.

System 10 can comprise at least two devices 100 that are implanted proximate an artery, such as a device 100 that is implanted proximate a radial artery (e.g. device 100i or 100j), a device 100 that is implanted near a carotid artery (e.g. device 100b), a device that is implanted proximate a brachial artery (e.g. device 100c), and/or at least a device 100 that is implanted proximate a posterior auricular artery (e.g. device 100a implanted behind the patient's ear). System 10 can comprise one, two, or more devices 100 that are implanted in the chest of the patient (e.g. devices 100d and 100e shown). In some embodiments, system 10 comprises at least two, or at least three devices that are implanted in the chest of the patient (e.g. to record heart activity, lung activity, or both). System 10 can comprise a device 100 that is implanted in the abdomen of the patient (e.g. device 100f and/or 100h shown), such as when the patient is a pregnant woman, such as to monitor fetal heart sounds. System 10 can comprise one, two, ore more devices implanted proximate a joint of the patient (e.g. device 100g implanted proximate an elbow, devices 100l and 100j implanted proximate a wrist, and/or device 100k implanted proximate a knee), such as to record joint movement sounds (e.g. to record sounds related to a tear or other injury).

In some embodiments, system 10 comprises a first device 100 implanted proximate a wrist of the patient (e.g. device 100i and/or 100j as shown), as well as a second device 100 implanted at a location remote from the patient's wrist, such as to record patient data with a wide vector collectively from both devices 100.

Referring now to FIG. 5, a top view of two electronic assemblies of an implantable device 100 are illustrated, consistent with the present inventive concepts. Each implantable device 100 can comprise electronic assemblies, such as circuit boards 190a and 190b shown. Electronic assemblies 190a,b can comprise various componentry configured to provide control assembly 160 described herein. In some embodiments, electronic assemblies 190a,b each comprise one side of a single printed circuit board assembly.

Electronic assembly 190a can comprise processor 165, memory 168 (e.g. memory that at least stores instructions for algorithm 166 described herein), and/or power supply 162 (e.g. a battery and/or a capacitor), each as shown. Electronic assembly 190a can comprise one or more sensors, such as sensor 120 shown. Sensor 120 can comprise an accelerometer, accelerometer 150 shown.

Electronic assembly 190b can comprise magnetic sensor 120a as shown. Magnetic sensor 120a can be configured to detect the presence of a magnet (e.g. a functional element 99 comprising a magnet), such that when the magnet is positioned (e.g. by the patient or a clinician of the patient) at a location external to the patient but proximate device 100 (e.g. proximate the implantation site of device 100), device 100 changes operational modes (e.g. device 100 starts recording data, stops recording data, and/or performs another operational mode change). For example, the patient can place the magnet proximate the device 100 if the patient suspects the presence of an arrhythmia or other event in which data recording should be initiated.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the present inventive concepts, which is defined in the accompanying claims.

1. A system for producing a diagnostic result for a patient comprising:
at least one implantable device configured to record physiologic data of the patient;
at least one external device configured to:
transmit power and/or data to the at least one implantable device; and/or
receive data from the at least one implantable device,
wherein the system is configured to produce the diagnostic result based on the recorded physiologic data.

2.-21. (canceled)
